(19) United States
(12) Patent Application Publication
(10) Pub. No.: US 2021/0398607 A1
(43) Pub. Date: Dec. 23, 2021

(54) METHODS FOR DESIGNING ANTIBODY SMALL-MOLECULE CONJUGATES

(71) Applicant: California Institute of Technology, Pasadena, CA (US)

(72) Inventors: Stephen L. Mayo, Pasadena, CA (US); Jingzhou Wang, Pasadena, CA (US); Aiden J. Aceves, Pasadena, CA (US)

(21) Appl. No.: 17/349,785

(22) Filed: Jun. 16, 2021

Related U.S. Application Data
(60) Provisional application No. 63/040,379, filed on Jun. 17, 2020.

(51) Int. Cl. G16B 15/30; A61K 47/68

(52) U.S. Cl. CPC G16B 15/30; A61K 47/6803

(57) ABSTRACT

Disclosed herein include methods, compositions, and systems for designing antibody-small-molecule conjugates, for example antibody-drug conjugates (ADCs). The designed antibody-small-molecule conjugates can have, for example, higher binding affinities to the target protein compared to the small molecule alone or to a reference antibody-small-molecule conjugate (e.g., the parent antibody-small-molecule conjugate).

### RELATED APPLICATIONS

The present application claims priority to U.S. Provisional Application No. 63/040,379, filed Jun. 17, 2020. The contents of this related applications is hereby expressly incorporated by reference in its entirety for all purposes.

### REFERENCE TO SEQUENCE LISTING

The present application is being filed along with a Sequence Listing in electronic format. The Sequence Listing is provided as a file entitled 30KJ_302433_US_sequence_listing, created Jun. 16, 2021, which is 22 kilobytes in size. The information in the electronic format of the Sequence Listing is incorporated herein by reference in its entirety.

### BACKGROUND

### Field

The present disclosure relates generally to the field of molecular biology, and more specifically designing of protein-small molecule conjugates.

### Description of the Related Art

Most pharmaceutical mechanisms involve drug-target interactions that are mediated by synthetic small molecules or monoclonal antibodies—the two major drug modalities. Until now, many biological pathways are still difficult to be pharmaceutically intervened, often because through existing approaches either the desired interactions are fundamentally difficult to be engineered, or the pharmacological trade-offs for establishing the interactions outweigh potential benefits. Therefore, new modalities that incorporate new chemistry and new biology are constantly created to realize a versatile toolkit that more easily tackles certain challenging targets, and also expands the targetable molecular space itself. One way for creating new modalities is combining existing modalities to consolidate individual advantages and offset individual flaws. Antibody-drug conjugates (ADCs), for example, takes advantage of the excellent specificity and biological compatibility of monoclonal antibodies to improve therapeutic indices of existing small-molecule drugs. Traditionally, the antibody and drug components of ADCs are separately developed and bind to different targets while in action. Most current ADCs improve the specificity of conjugated drugs as they deliver the small molecules into cell targets through specific antibody-induced receptor endocytosis. Some ADCs and peptide-drug conjugates were also reported to improve the metabolic stability, circulation half-life, and solubility of linked small molecules through antibody-associated pharmacokinetics, chemical environment around the conjugation sites, and linker design, indicating that protein conjugation could modulate a wide range of small-molecule properties.

To engineer the binding synergy required for this kind of application, current methods that separately develop and characterize the antibody and small molecule components would be resource intensive, thus limiting the application scenarios. There is a need for methods for rationally-designed antibody conjugation to optimize the mechanism of action, along with many other pharmacologically-relevant properties, of small-molecule based binders.

### SUMMARY

Disclosed herein include a method for designing antibody-small-molecule conjugates. The method can include, for example, (a) receiving three-dimensional coordinates for a crystal structure of a target protein in complex with a small molecule; (b) docking a plurality of antibody structures onto the crystal structure, wherein each of the plurality of antibody structures has a different complementarity-determining region (CDR) conformations from each other and thereby different binding pose against the target protein surface; (c) identifying one or more of the plurality of antibody structures with binding poses that accommodate both the CDR binding poses and the target protein-small molecule interaction; (d) screening a rotamer library of the conjugated small molecule onto the CDR binding poses identified in (c) to identify a conjugation plan comprising a selected conjugation site on the antibody to which the small molecule is conjugated to; and (e) adjusting the sequences of the antibody CDR loops, the antibody framework or both in the conjugation plan identified in (d) to generate an antibody capable of forming an antibody-small-molecule conjugate with the small molecule, wherein the antibody-small-molecule conjugate has a higher binding affinity to the target protein as compared to the small molecule alone or a reference antibody-small-molecule conjugate to the target protein. In some embodiments, identifying the antibody structures in step (c) comprises performing loop-modeling on docketed poses. In some embodiments, performing loop-modeling on docketed poses comprises searching naturally occurring antibody CDR binding conformation.

The binding affinity of the antibody-small-molecule conjugate to the target protein can be higher, for example at least two fold higher or at least five folder higher, than the binding affinity of the small molecule alone or the reference antibody-small-molecule conjugate. In some embodiments, the target protein-small molecule interaction in (c) comprises an interaction between the small molecule and a variant of the target protein. In some embodiments, the rotamer library of the conjugated small molecule is a rotamer library of cysteine-conjugated side chain. In the method, adjusting the sequence of the antibody CDR loops in (e) can, for example, comprise adjusting the sequence of the antibody CDR residues close to the binding sites of the small molecule to the protein target. Non-limiting examples of the antibody include a nanobody, a monoclonal antibody, and a combination thereof. The antibody-small-molecule conjugate can, for example, have one or more improved properties, including but not limited to kinetics, metabolic stability, circulation half-life, solubility, systemic toxicity, or any combination of, compared to the small molecule alone. In some embodiments, the antibody-small-molecule conjugate has improved binding specificity to the protein target compared to the small molecule alone.

The method can, in some embodiments, further comprises adjusting the sequences of the antibody CDR loops to increase H-bond formation between the antibody and the target protein surface. In some embodiments, the binding surface for the designed antibody-small-molecule conjugate to the target protein comprises an ultra-deep pocket, broad contacting interface, or both. The small molecule can be, for example, a therapeutic agent (including but is not limited to, a cancer drug or a cytotoxic drug). In some embodiments, the target protein is a tumor antigen. In some embodiments, the antibody-small-molecule conjugate is an antibody-drug conjugate (ADC), for example, Gemtuzumab ozogamicin, Brentuximab vedotin, Trastuzumab emtansine, Inotuzumab ozogamicin, Polatuzumab vedotin, Enfortumab vedotin, Trastuzumab deruxtecan, Sacituzumab govitecan, Belantamab mafodotin, Moxetumomab pasudotox, or Loncastuximab tesirine. In some embodiments, two or more of the plurality of antibody structures have different CDR sequences. Also provided includes a method for producing one or more of the designed antibody-small-molecule conjugate using the method disclosed herein.

### BRIEF DESCRIPTION OF THE DRAWINGS

The patent or application file contains at least one drawing executed in color. Copies of this patent or patent application publication with drawing(s) will be provided by the Office upon request and payment of the necessary fee.

FIG. 1A-FIG. 1F depict data showing computationally-designed nanobody-biotin conjugates bind stronger than biotin itself against mSA streptavidin. For SPR-measured KD, kₒₙ, and k_{off} results, data from one of the triplicates is shown here, and data from the other two replicates is in FIG. 11A-FIG. 11J. FIG. 1A shows a non-limiting schematic representation of an exemplary design workflow described herein. Given the availability of a small molecule and its target, the sequence of a complementary immunoglobulin domain and a conjugation plan with the small molecule are computationally determined to create conjugates that synergistically bind to the target. FIG. 1B depicts a finalized model of 4NBX.B-biotin103 in complex with mSA streptavidin. The mSA is colored green, and nanobody scaffold is colored cyan. Biotin103 side chain is shown as stick, and the H-bond forming potential of Y112 and R27 with mSA residues is also represented. FIG. 1C depicts data showing Y112 and R27 are predicted to participate in a broader potential H-bond network that involves biotin/mSA interactions. FIG. 1D shows SPR estimation of mSA/biotin binding parameters by two methods. FIG. 1E shows SPR measurements determining that 4NBX.B-biotin103 occupies the biotin-binding pocket of mSA with improved affinity and kinetics. FIG. 1F shows SPR measurements determining that 4NBX.B_biotin103 binds stronger towards a weaker biotin-binding mutant of mSA than biotin itself.

FIG. 2A-FIG. 2B show that CDR sequence design enhanced the mSA-binding affinity and kinetics of 4NBX.B-biotin103. In the structural models, the mSA is colored green, and nanobody scaffold is colored cyan. Biotin103 side chain is shown as stick. For SPR-measured K_{D}, kₒₙ, and k_{off} results, data from one of the triplicates is shown here, and data from the other two replicates is in FIG. 11A-FIG. 11J. FIG. 2A shows predicted affinity-contributing mutations and SPR-measured binding profiles of 4NBX.B-biotin103 v119 against mSA_{WT}. FIG. 2B shows predicted affinity-contributing mutations and SPR-measured binding profiles of 4NBX.B-biotin103 v149 against mSA_{WT}.

FIG. 3A-FIG. 3C show that CDR sequence designed followed by framework design monomerically stabilized the designed conjugates without imposing affinity penalty. FIG. 3A shows SEC traces of biological triplicates (colored by blue with different intensity) for designed 4NBX.B-biotin103 conjugates, normalized by monomer peak height for better comparison of aggregates formation. SEC trace of 4NBX.B WT is overlaid with 4NBX.B-biotin103 WT traces and colored orange. Air-bubble peaks are marked by *. SEC trace of 4NBX.B WT nanobody was overlaid with 4NBX.B-biotin103 WT traces as reference. FIG. 3B shows SPR-measured binding profile of v186 and v186_Fr against mSA_{WT} indicates that the improved binding affinity and kinetics in v149 are preserved. For v186_Fr, data from one of the triplicates is shown here, and data from the other two replicates is in FIG. 11A-FIG. 11J. FIG. 3C shows a structural representation of nanobody amino acid position 12 and 37 before and after framework redesign. v186 is colored as green, and v186_Fr is colored as cyan. Additional H-bonds introduced by F37Y with CDR3 residues are shown as dashes, while the relevant CDR3 residues are also shown in both v186 and v186_Fr models.

FIG. 4A-FIG. 4E shows that MD simulation reveals design flaws and validates design success. FIG. 4A shows that MD simulation revealed possible origins for the improved monomeric stability of 4NBX.B-biotin103 v186_Fr compared to v186. Here shows the analysis of the solvent-accessible area for the selected hydrophobic residues of v186 and v186_Fr from 100 ns MD simulations performed in triplicates. The selected residues are presented as spheres in the nanobody models shown on both panels. The observed distributions of the solvent accessible area for the selected residues from the 3× simulations of v186 and v186_Fr are plotted into 80 bins along the x-axis (bars) with respective kernel density estimation (lines). Top panel: analysis of the hydrophobic core residues. Bottom panel: analysis of the CDR3-shielded residues. FIG. 4B-FIG. 4E show analysis of the interaction interface in the triplicate MD simulations of 4NBX.B-biotin103 v186_Fr against mSA_{WT}. Traces from simulation replicates are plotted on top of each other along the 100 ns timescales. Changes of whole-structure RMSD (FIG. 4B), interface shape complementarity (FIG. 4D), buried surface area (FIG. 4C), and interface separation distance along the time trajectories (FIG. 4E) are plotted.

FIG. 5A-FIG. 5D show summary of the design results for 4NBX.B-biotin103 and the overall design workflow. FIG. 5A-FIG. 5B show summary of SPR-measured binding affinity and kinetics of 4NBX.B-biotin103 conjugates and controls against mSA_{WT} and mSA_{S27A}. Experimental designs for each binding experiment are depicted by the cartoon above the lanes. Blue square: SPR cheap for immobilization. Spheres: molecules immobilized (attached to chip) or flew through (floating above the chip) during binding experiments. Different molecules are represented by different colors. Individual data points represent measurements from biological triplicates. Error bars represent standard deviation. FIG. 5C shows SPR-measured binding profile of the best-designed 4NBX.B-biotin103 variant v183_Fr against mSA_{S27A}, the weak-biotin-binding variant. Data from one of the triplicates is shown here, and data from the other two replicates is in FIG. 11A-FIG. 11J. FIG. 5D shows summary of a non-limiting exemplary design workflow for synergistically-binding nanobody-small molecule conjugates.

FIG. 6A-FIG. 6D show designed model, binding profiles, and aggregation profiles of a non-limiting exemplary nanobody scaffold obtained by directly docking against mSA. FIG. 6A shows a designed binding model and conjugation scheme of 2X89.A-CCAA-biotin57 against mSA streptavidin surface. FIG. 6B shows that SPR-measured binding profile of 2X89.A-CCAA-biotin57 against mSA_{WT} indicates an especially slow dissociation rate. Data from one of the triplicates is shown here, and data from the other two replicates is in FIG. 11A-FIG. 11J. FIG. 6C shows affinity and kinetics profile summary. FIG. 6D shows size-exclusion chromatography (SEC) traces of biological triplicates for 2X89.A-CCAA-biotin57, normalized by monomer peak height for better comparison of aggregates formation. Air-bubble peaks are marked by *.

FIG. 7A-FIG. 7E show determination of optimal CDR conformation against monomeric streptavidin model. FIG. 7A shows whole structural-alignment of 154 curated PDB nanobody scaffolds with diverse CDR conformations and sequences. FIG. 7B shows interface statistics of naturally occurring nanobody-target complexes. Error bars represent standard deviations. FIG. 7C-FIG. 7E depict non-limiting exemplary 7 docked poses of nanobody scaffolds that passed the filters selecting poses that most likely recapitulate the natural binding modes of the corresponding nanobody scaffolds. Streptavidin S45A/T90A/D180A is colored green, and nanobody scaffold is colored cyan.

FIG. 8A-FIG. 8D show identification of optimal conjugation strategy and finalized conjugate models. FIG. 8A shows that biotin conjugation was performed by biotin C2 maleimide with mutated cysteine residues. FIG. 8B shows the prepared structure of 4NBX.B-biotin103 in complex to streptavidin S45A/T90A/D180A. Streptavidin S45A/T90A/D180A is colored green, and nanobody scaffold is colored cyan. FIG. 8C shows that the H-bond forming potential of Y112 and R27 in 4NBX.B nanobody was predicted to be recapitulated in the designed binding pose with the streptavidin model. Streptavidin S45A/T90A/D180A is colored green, and nanobody scaffold is colored cyan. Biotin103 side chain is shown as stick. Y112 and R27 together with their predicted H-bond partners are shown as line. FIG. 8D shows alignment results for prepared structures of 4NBX.B-biotin103 in complex with streptavidin S45A/T90A/D180A and mSA. Streptavidin models are colored green, and nanobody scaffolds are colored cyan. Residues that were identified by sequence alignment as pair-wise identical sequences are colored red. Biotin103 side chain from both models are shown as stick.

FIG. 9A-FIG. 9B show additional exemplary supporting SEC traces. FIG. 9A SEC traces of mSA_{WT} and mSA_{S27A}. FIG. 9B shows SEC rerun trace of collected monomeric fraction for 4NBX.B-biotin103 v186_Fr. The air bubble peak is labeled as *.

FIG. 10A-FIG. 10B shows a summary of MD simulations performed for 4NBX.B-biotin103 v186 and v186_Fr against mSA_{WT}. For each simulation, 400 snapshots evenly spaced along the 100 ns timescale are aligned together. green: mSA_{WT}. cyan: nanobody-biotin conjugates. The biotin103 “residue” is shown as stick.

FIG. 11A-FIG. 11J show SPR measurements from additional exemplary biological replicates included for affinity and kinetics estimation.

FIG. 12A-FIG. 12C show a non-limiting exemplary illustration of intact-protein mass spectrometry (MS) workflow to assess the purity of nanobody conjugates, using the analysis results of 4NBX.B-biotin103 v119 prepared without TCEP reduction as an example. The liquid chromatography (LC) column is first washed with isopropyl alcohol to clean the column and reveal background peaks that are irrelevant to the samples of interest. The nanobody conjugates sample is then applied to the LC-MS. Note that all nanobodies being analyzed are eluted in a single LC peak, of which the MS spectrum is de-convoluted to review the molecular weights of the components. Monomeric and dimeric nanobody impurities are successfully identified after de-convolution, exemplified here by the identified single glutathione-modified nanobody and disulfide-linked nanobodies in the 4NBX.B-biotin103 v119 conjugate that was prepared without TCEP reduction to expose all of the engineered CDR cysteine.

FIG. 13A-FIG. 13D show that intact-protein mass spectrometry (MS) confirmed mono-conjugated materials. MS deconvolution of nanobody-biotin conjugates only returned MWs within 20 Da from expected MW of mono-conjugated materials.

FIG. 14A-FIG. 14B show exemplary structural representation and binding curve against mSA_{WT} for the +1 and −1 variants of 4NBX.B-biotin103 v186_Fr.

FIG. 15A-FIG. 15G show construction and testing of a sequence design pipeline. FIG. 15A depicts a non-limiting exemplary rudimentary sequence design pipeline that automatically performs CDR and framework design in a step wise manner. FIG. 15B shows outputs from the pipeline illustrated in FIG. 15 A and the number of accumulated mutations. FIG. 15C shows predicted H-bond formation profile shifts to different locations in variant v5 compared to the wild type conjugate. FIG. 15D-FIG. 15G show SEC traces, newly-accumulated mutations, and SPR binding curves for the designed variants of 2X89.A-CCAA-biotin57.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Conjugates of small molecule drugs with antibodies (ADCs) have been developed as a promising class of targeted therapeutics combining the specificity of antibodies (e.g., monoclonal antibodies (mAbs)) with potent cytotoxic activity of small molecule drugs for the treatment of cancer and other diseases. Recently, advances in identifying targets, selecting highly specific antibodies of preferred isotypes, improving methods for conjugation have led to the FDA approval of a number of ADCs and many other ADCs in advanced clinical development. However, the complex and heterogeneous nature of ADCs can cause, for example, poor solubility, instability, aggregation, affinity and unwanted toxicity. Disclosed herein include methods of designing antibody-small-molecule conjugates that with more desired properties than the small molecule alone in binding to the target protein. The methods can also be used in designing antibody-small-molecule conjugates that have improved properties, such as kinetics, metabolic stability, circulation half-life, solubility, systemic toxicity, or any combination of, compared to the small molecule alone. Also provided herein include producing the antibody-small-molecule conjugates designed using the methods disclosed herein, the antibody-small-molecule conjugates produced and compositions thereof, and the methods of using the antibody-small-molecule conjugates and the compositions in treating subjects in need thereof.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In some embodiments, an ADC includes an antibody, a cytotoxic or therapeutic drug, and optionally a linker that enables attachment or conjugation of the drug to the antibody. An ADC can have, for example, from 1 to 8 drugs conjugated to the antibody, including drug loaded species of 2, 4, 6, or 8. Non-limiting examples of drugs that can be included in the ADCs include mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, radiosensitizers, or any combination thereof.

As used herein, the term “antibody” broadly refers to an immunoglobulin (Ig) molecule, generally comprised of four polypeptide chains, two heavy (H) chains and two light (L) chains, or any functional fragment, mutant, variant, or derivative thereof, that retains the essential target binding features of an Ig molecule. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY) and class (e.g., IgG1, IgG2, IgG 3, IgG4, IgA1 and IgA2) or subclass. The antibody can be, for example, an intact antibody, a monoclonal antibody, an antibody fragment, a chimeric antibody, a humanized antibody, diabodies, a nanobody, or a combination thereof.

As used herein, the term “chemotherapeutic agent” refers to a compound (e.g., a small molecule) useful in the treatment of cancer.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems, including but not limited to, Kabat CDRs and Chothia CDRs.

As used herein, the term “cancer” refers a physiological condition in mammals that is typically characterized by unregulated cell growth. Non-limiting examples of cancer include carcinoma, lymphoma, blastoma, sarcoma, and leukemia or lymphoid malignancies. More particular examples of such cancers include glioblastoma, non-small cell lung cancer, lung cancer, colon cancer, head and neck cancer, breast cancer, squamous cell tumors, anal cancer, skin cancer, and vulvar cancer. In some embodiments, the cancer is solid tumor.

Disclosed herein includes methods, compositions and systems capable of efficiently determining a compatible antibody sequence and conjugation strategy for a to-be-improved small molecule binding event.

The computationally-designed synergistically-binding antibody small-molecule conjugates generated using the methods disclosed herein can have CDR loops chemically extended beyond the natural repertoire, and are named are named CDR-extended antibody, abbreviated as CDRexAb.

Computational protein design (CPD) refers to a series of in silico methods that use sample amino acid sequences and conformational space to generate polypeptide chains with desired structure, function, or both. Disclosed herein include methods for using CPD to design antibody-small-molecule conjugates which are chimeric molecules created by combining full-length or fragments of antibodies with organic compounds (e.g., small molecule drugs) that bind to molecular targets (e.g., tumor antigens).

As described herein, CPD can be used to design CDR-extended antibodies: antibody small-molecule conjugates whose amino acid sequences synergize with the conjugated small molecules to bind cooperatively to a shared surface on a molecular target, which can also be recognized by the small molecule alone. One of the advantages of the methods disclosed herein is that the design process can be initiated using, for example, only the structural detail of the small molecule interacting with the target protein (e.g., three-dimensional coordinates for a crystal structure of the target protein in complex with the small molecule). The design can be used, for example, to generate synergistically-binding conjugates that have different binding properties (e.g., bind tighter to the protein target than the small molecule alone, or binding tighter to the protein target than a reference antibody-small-molecule conjugate). The design can also be used to, for example, generate antibody-small-molecule conjugates that possess improved pharmacologically-relevant properties, including but not limited to solubility, stability, and serum half-life than the small molecule alone, or a reference antibody-small-molecule conjugate.

In some embodiments, the method of designing antibody-small-molecule conjugates (e.g., antibody-small-molecule conjugates with desired properties) includes one or more of the following steps 1-4:

1. In silico designing new antibody-target binding conformations: antibody scaffolds with diverse conformations and sequences are directly curated from public databases of protein and antibody structures. Optimal binding conformations of the diverse repertoire of antibody scaffolds are first searched by docking the scaffolds against the target surface with the small molecule already modeled in, then scored, and finally filtered.

2. Designing the optimal conjugation strategy for the small molecule: based on the finalized antibody binding conformation, the small molecule with a selected linker arm can be diversified to create a range of possible conformations. Each generated conformer can be computationally screened against each residue of the complementarity-determining region (CDR) loops of the modeled antibody to identify (1) conjugation sites with optimal bond-forming favorability with the low-energy conformer of the small molecule-linker arm and (2) conjugation sites with minimal clash with the small molecule-linker arm.

3. Sequence optimization of the antibody: after making the conjugated model based on the optimal antibody scaffolds, binding poses, and conjugation sites in the previous two steps, the sequence of the antibody is computationally optimized at both the CDR loop regions and constant framework regions. Top design results can be scored, filtered, and either individually tested as ultra-small protein libraries or combined to design larger but defined combinatorial libraries that are more suitable to be tested by display-based screening and selection techniques.

4. Experimental validation: binding affinity and other properties of interest for the designed conjugates can be experimentally tested to validate successful designs.

In some embodiments, CDR-extended antibodies and their design workflow can be used to optimize small-molecule based binders in diagnostics and pharmaceuticals-related applications. Examples of specific application include but are not limited to as follows:

1. Given a low-affinity small molecule binder, design it into a CDR-extended antibody to exhibit improved affinity against its target molecule.

2. Given a failed small molecule drug candidate, reutilize it by designing it into a CDR-extended antibody that fixes the disadvantageous properties behind the drug failure.

3. Given a particularly challenging target surface that is difficult for both small molecules and antibodies alone, designing CDR-extended antibodies that incorporate small molecule fragments that cannot function as good binders alone, but are more compatible with the target of interest than the 20 canonical amino acids.

Compared to existing methods for the above-mentioned application scenarios, CDR-extended antibody has various advantages, including but not limited to: (1) it includes large proteins into optimizing binding events that involve synthetic small molecules, therefore brings in a chemical space that has not been explored before; (2) the binding interface for CDR-extended antibodies contain both deep pocket that is uncommon for antibodies, and broad contacting interface that is uncommon for small molecules, therefore a new target space can also be explored. Given the fact that still only a small fraction of the human proteome is currently druggable, a method that expands both the target space and chemical space of molecular recognition agents would be highly beneficial; and (3) compared to traditional antibody-drug conjugates that explore the modular applications of existing molecules, CDR-extended antibodies explore the sub-molecular and atomic-level cooperation of different modalities, further bridging small and large molecules.

In some embodiments, generating CDR-extended antibodies can further comprise: (1) using existing structures of small molecules and targets as input, as well as docked structures of small molecule against targets; (2) random mutagenesis to further improve the designed CDR-extended antibodies; (3) improve binding affinity to the target protein, specificity to the target protein, or both, for the conjugates; and (4) using existing structures of antibody scaffolds, as well as structures diversified by computational methods such as structural recombination and molecular dynamics, as input for binding pose optimization.

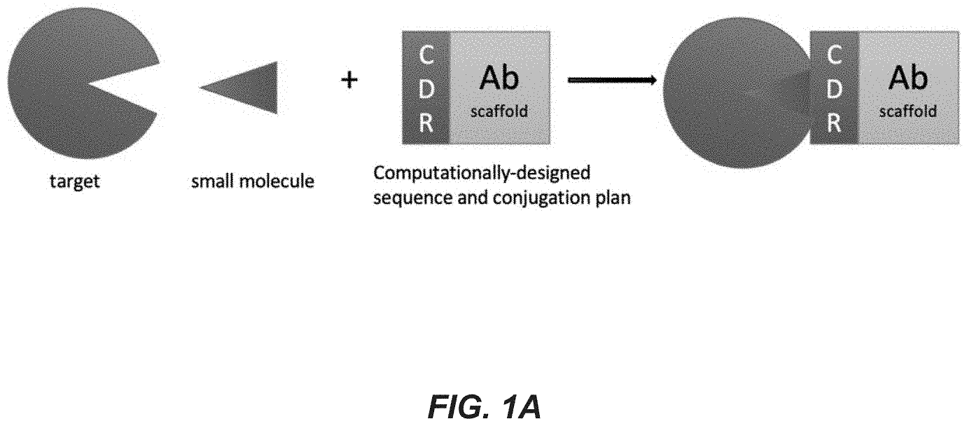

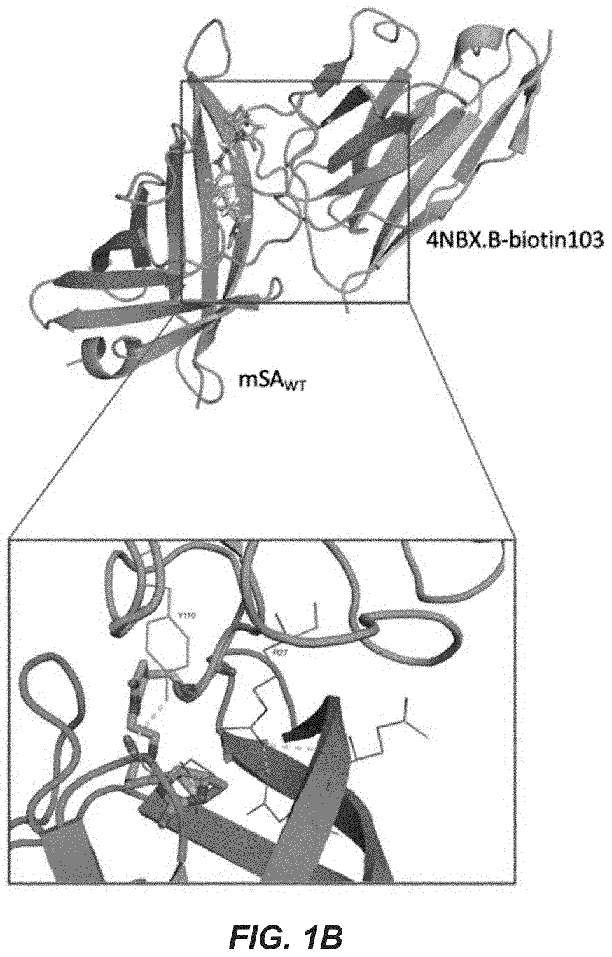

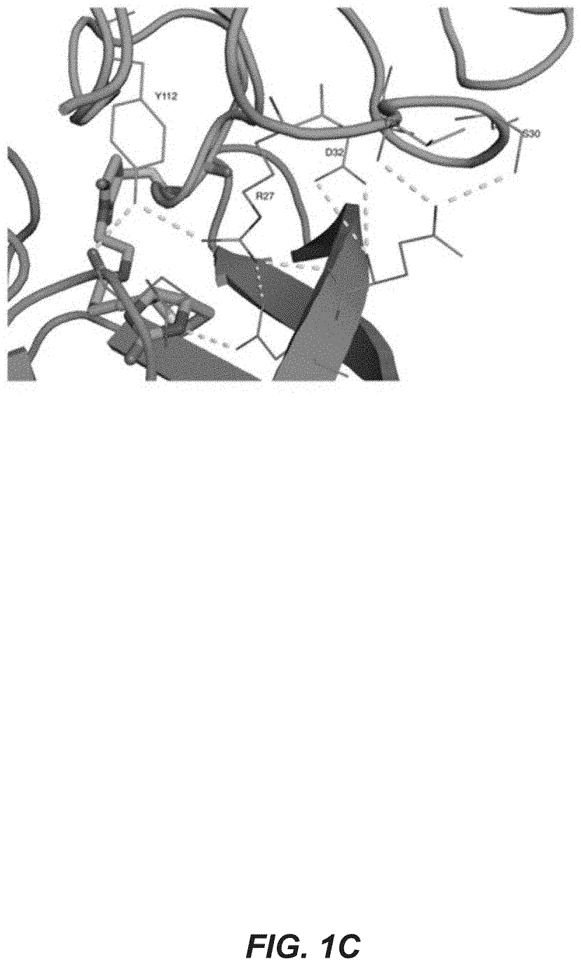

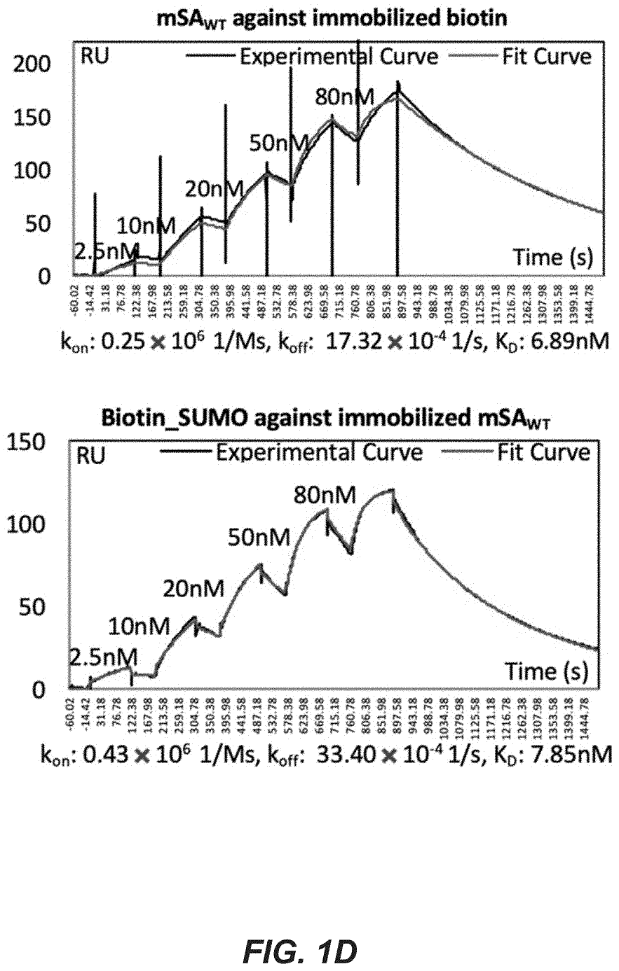

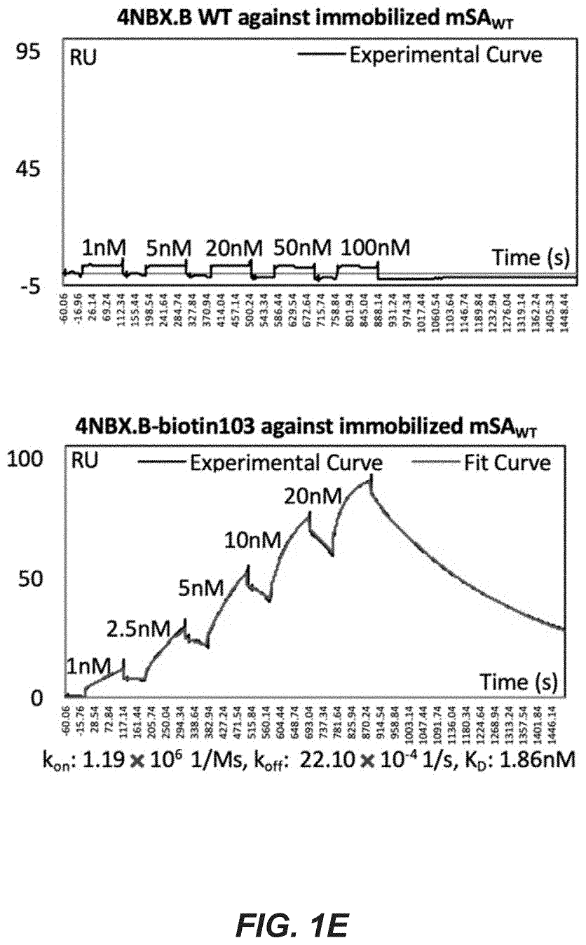

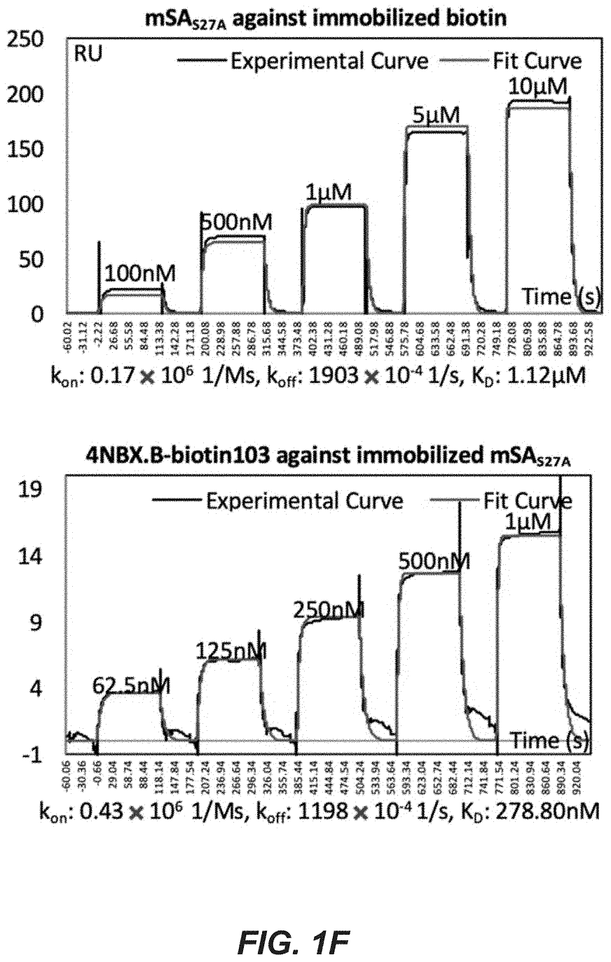

The methods disclosed herein enables computationally designing the antibody component of synergistically-binding ADCs. The concept of CDR-extended antibodies (CDRexAbs) is described herein, which refers to computationally-designed antibodies whose complementary-determining regions (CDRs) contain a small molecule ligand that binds to a certain target, with surrounding CDR sequences tailored to strengthen the target-binding interactions (FIG. 1A). As described herein, the design was focused on nanobodies, which are llama-derived single-domain antibody fragments that can function by themselves, with attached Fc domains, or reformatted into IgGs. Using a modified streptavidin-biotin interaction pair as model system, it was demonstrated that with only the structural knowledge of small-molecule/target interactions, nanobody small-molecule conjugates can be computationally designed to bind tighter against the target than the small molecule itself. Through subsequent sequence design, the affinity, binding kinetics, and overall stability of the conjugates can be improved in a step-wise manner. ≥20-fold affinity improvements together with targeted kinetic-tuning can be achieved when the starting small-molecule/target affinity is as weak as 1 μM, or as strong as 7 nM. Exploration of various computational methods revealed key design principles, from which a general design strategy was proposed for this new potential modality.

Disclosed herein includes a method for designing antibody-small-molecule conjugates. The method comprises, in some embodiments: step (a) receiving three-dimensional coordinates for a crystal structure of a target protein in complex with a small molecule; step (b) docking a plurality of antibody structures onto the crystal structure, wherein each of the plurality of antibody structures has a different complementarity-determining region (CDR) conformations from each other and thereby different binding pose against the target protein surface; step (c) identifying one or more of the plurality of antibody structures with binding poses that accommodate both the CDR binding poses and the target protein-small molecule interaction; step (d) screening a rotamer library of the conjugated small molecule onto the CDR binding poses identified in step (c) to identify a conjugation plan comprising a selected conjugation site on the antibody to which the small molecule is conjugated to; and step (e) adjusting the sequences of the antibody CDR loops, the antibody framework or both in the conjugation plan identified in step (d) to generate an antibody capable of forming an antibody-small-molecule conjugate with the small molecule. In some embodiments, the antibody-small-molecule conjugate has a higher binding affinity to the target protein as compared to the small molecule alone. In some embodiments, the antibody-small-molecule conjugate has a higher binding affinity to the target protein as compared to a reference antibody-small-molecule conjugate, for example the parent antibody-small-molecule conjugate for which the designed antibody-small-molecule conjugate is a variant of. In some embodiments, two or more of the plurality of antibody structures have different CDR sequences. In some embodiments, each of the plurality of antibody structures has different CDR sequence from each other.

Various methods are currently available to determine the three-dimensional coordinates for the crystal structure of a protein alone or a protein in complex with another biological entity (such as a small molecule). For example, the three-dimensional coordinates for the crystal structure of a protein in complex with a small molecule can be determined by nuclear magnetic resonance (NMR) spectroscopy, X-ray crystallography, or both. The structures of many proteins had been elucidated by NMR and x-ray crystallography, and the coordinates are collected at, for example, the Protein Data Bank (http://www.rcsb.org/pdb) and the structures can be accessed for visualization and analysis. In some embodiments, the method disclosed herein includes receiving three-dimensional coordinates for a crystal structure of the target protein in complex with a small molecule determined by X-ray crystallography. In some embodiments, the three-dimensional coordinates for a crystal structure of the target protein in complex with a small molecule are obtained from the Protein Data Bank.

The methods described herein can include docking a plurality of antibody structures onto the crystal structure, wherein each of the plurality of antibody structures has a different complementarity-determining region (CDR) conformations from each other and thereby different binding pose against the target protein surface. Protein-protein docketing is a technique capable of predicting the structure of proteins, protein-protein complexes, or bound structure of proteins in complex with other biological entities (such as small molecules), given the structures of known structures (e.g., experimentally determined protein structures). In some embodiments, the known protein structures are high-resolution structures determined experimentally by x-ray crystallography. In some embodiments, the known protein structures are lower-resolution modeled structures. Docking approaches can take into consideration of the conformational changes between unbound and bound structures, as well as the inaccuracies of the interacting modeled structures. Non-limiting exemplary methods for protein-protein docketing include SwarmDock (a flexible docking method which uses a population-based memetic algorithm to optimize parameters characterizing the orientation, position, and conformations of protein subunits), pepATTRACT, FlexPepDock, HADDOCK, and PEP-SiteFinder. Various molecular docking methods for protein-peptide docking are described and compared in a study described in Agrawal et al. (“Benchmarking of different molecular docking methods for protein-peptide docking,” BMC Bioinformatics 2019, 19(Suppl 13):426, the content of which is incorporated by reference). In addition, the ClusPro server (https://cluspro.org) is a widely used tool for protein-protein docking. It is contemplated that any technique suitable for protein-protein/protein-peptide docking can be used in the methods described herein, so that one or more of the plurality of antibody structures with binding poses that accommodate both the CDR binding poses and the target protein-small molecule interaction can be identified. In some embodiments, identifying the antibody structures with binding poses that accommodate both the CDR binding poses and the target protein-small molecule interaction comprises performing loop-modeling on docketed poses. In some embodiments, performing loop-modeling on docketed poses comprises searching naturally occurring antibody CDR binding conformation.

The antibody structures that are used for docking onto the crystal structure can be, for example, curated PDB antibody (e.g., nanobody) scaffolds. The PDB antibody scaffolds can have diverse CDR confirmations and sequences. The docking can, for example, comprise determining interface statistics of the complex. The docking can, in some embodiments, comprise obtaining interface statistics (e.g., interface separation distance, buried interface area, shape complementarity score, or a combination thereof) of known antibody-target protein complexes (e.g., nanobody-target protein complexes), and/or comparing the interface statistics of the known antibody-target protein complexes with the docked structures/complexes. The known antibody-target protein complexes can be, for example, naturally occurring antibody-target protein complexes. Using the methods disclosed herein, two or more (for example, 2, 3, 4, 5, 6, 7, 8, 9, 10, 11, 12, 13, 14, 15, 16, 17, 18, 19, 20, or a number or a range between two of these values) antibody structures with binding poses that accommodate both the CDR binding poses and the target protein-small molecule interaction can be identified. In some embodiments, a plurality of antibody structures (e.g., a nanobody scaffolds) can be ranked for its CDR conformation against known antibody structure model (e.g., ranked based on one or more interface statistics), and the one or more antibody structures that passes filters selecting poses are considered to be most likely to recapitulate the natural binding modes of the corresponding antibody structures (e.g., nanobody scaffolds), and identified as having binding poses that accommodate both the CDR binding poses and the target protein-small molecule interaction. In some embodiments, the binding surface for the designed antibody-small-molecule conjugate to the target protein comprises an ultra-deep pocket, broad contacting interface, or both.

Rotamer libraries of the conjugated small molecule (e.g., a cancer therapeutic agent) can be screened onto the CDR binding poses identified in the methods described herein (the identified binding poses are capable of accommodate both the CDR binding poses and the target protein-small molecule interaction) to identify a conjugation plan comprising a selected conjugation site on the antibody to which the small molecule is conjugated to. The rotamer library can be a backbone-dependent rotamer library which provides, for example, the frequencies, mean dihedral angles, and standard deviations of the discrete conformations (known as rotamers) of the amino acid side chains in proteins as a function of the backbone dihedral angles φ and ψ of the Ramachandran map (e.g., Dunbrack backbone-dependent rotamer library), or a backbone-independent rotamer library which can, for example, express the frequencies and mean dihedral angles for all side chains in proteins, regardless of the backbone conformation of each residue type. In some embodiments, it can be advantageous to use backbone-dependent rotamer libraries which when used as an energy term, can speed up search times of side-chain packing algorithms used in protein structure prediction and protein design. Additional non-limiting examples of rotamer library include Dynameomics rotamer library, Richardson (common-atom values) backbone-independent rotamer library, and Richardson (mode values) backbone-independent rotamer library. In some embodiments, the methods disclosed herein use a rotamer library providing multiple choices of type for cysteine and histidine depending on the oxidation or protonation state of the sidechain: CYH—cysteine reduced free sulfhydryl, CYS—cysteine oxidized disulfide-bonded (half-cystine), HID—histidine neutral δ-protonated, HIE—histidine neutral ε-protonated, HIS—histidine neutral (HID and HIE combined), and HIP—histidine positive protonated on both sidechain nitrogens. In some embodiments, the rotamer library of the conjugated small molecule is a rotamer library of cysteine-conjugated side chain.

The methods described herein can comprise adjusting the sequences of the antibody CDR loops, the antibody framework or both in the identified conjugation plan to generate an antibody capable of forming an antibody-small-molecule conjugate with the small molecule with different properties than the small molecule alone or than a reference antibody-small-molecule conjugate (e.g., a known antibody-small-molecule conjugate for the small molecule of interest). The designed antibody-small-molecule conjugate can, for example, have a higher binding affinity to the target protein as compared to the small molecule alone, or a higher binding affinity to the target protein as compared to a reference antibody-small-molecule conjugate. The binding affinity of the antibody-small-molecule conjugate to the target protein can be, for example, 50%, 75%, 100%, 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 15 fold, 20 fold, 25 fold, 30 fold, or a number or a range between any two of these values, higher than the small molecule alone or than the reference antibody-small-molecule conjugate. In some embodiments, the binding affinity of the antibody-small-molecule conjugate to the target protein can be, for example, at least or at least about, 50%, 75%, 100%, 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 15 fold, 20 fold, 25 fold, or 30 fold higher than the small molecule alone or than the reference antibody-small-molecule conjugate. The antibody-small-molecule conjugate can, for example, improved kinetics, metabolic stability, circulation half-life, solubility, systemic toxicity, or any combination of, compared to the small molecule alone or the reference antibody-small-molecule conjugate. For example, the antibody-small-molecule conjugate can have metabolic stability 50%, 75%, 100%, 1.5 fold, 2 fold, 2.5 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 15 fold, 20 fold, 25 fold, 30 fold, or a number or a range between any two of these values better than the small molecule alone or the reference antibody-small-molecule conjugate. In some embodiments, the antibody-small-molecule conjugate can have circulation half-life 25%, 50%, 75%, 100%, 125%, 150%, 200%, or a number or a range between any two of these values better than the small molecule alone or the reference antibody-small-molecule conjugate. In some embodiments, the antibody-small-molecule conjugate has improved binding specificity to the protein target compared to the small molecule alone or the reference antibody-small-molecule conjugate. For example, the antibody-small-molecule conjugate can have a binding specificity to the protein target 5%, 10%, 20%, 25%, 35%, 50%, or 75% better than the small molecule alone or the reference antibody-small-molecule conjugate.

As used herein, the term “target protein” refers to a protein of interest as well as variants of the protein. For example, a variant of a protein can possess one or more single amino acid substitutions, one or more amino acid deletions, one or more amino acid additions (all referred to as amino acid mutations) as compared to the protein. A variant of a protein can possess a sequence having, for example, 80%, 85%, 90%, 91%, 92%, 93%, 94%, 95%, 96%, 97%, 98%, 99%, or more, sequence identity to the protein. As described herein, in identifying one or more of the plurality of antibody structures with binding poses that accommodate both the CDR binding poses and the target protein-small molecule interaction, the target protein-small molecule interaction can be or comprises an interaction between the small molecule and a variant of the target protein.

The sequence of the antibody structures identified by the methods described herein with binding poses that accommodate both the CDR binding poses and the target protein-small molecule interaction can be adjusted, for example the sequence of the antibody CDR residues close to the binding sites of the small molecule to the protein target. The adjustment to sequence can include substitution(s), deletion(s) and/or addition(s) at one, two, three, four, five, six, seven, eight, nine, ten, or a number or a range between two of these values, amino acids. In some embodiments, the sequence adjustment is to the one, two or three amino acids closest on the antibody to the binding sites of the small molecule to the protein target. In some embodiments, the sequence adjustment is to the one, two or three amino acids closest on the CDRs of the antibody to the binding sites of the small molecule to the protein target, further comprising adjusting the sequences of the antibody CDR loops to increase H-bond formation between the antibody and the target protein surface.

The type of small molecule in the antibody-small-molecule conjugates that the methods described herein can be used to design can vary. For example, the small molecule is can be a therapeutic agent, including but not limited to a cancer drug or a cytotoxic drug. The type of target protein for the antibody-small-molecule conjugates that the methods described herein can be used to design can vary. For example, the target protein can be a tumor antigen, In some embodiments, the antibody-small-molecule conjugate is an antibody-drug conjugate (ADC). Non-limiting examples of the ADC include Gemtuzumab ozogamicin, Brentuximab vedotin, Trastuzumab emtansine, Inotuzumab ozogamicin, Polatuzumab vedotin, Enfortumab vedotin, Trastuzumab deruxtecan, Sacituzumab govitecan, Belantamab mafodotin, Moxetumomab pasudotox, and Loncastuximab tesirine.

Also provided include producing one or more of the antibody-small-molecule conjugates designed using the methods described herein, using the designed antibody-small-molecule conjugates to contact disease cells (e.g., cancer cells) to determine their therapeutic effects and/or efficacies, using the designed antibody-small-molecule conjugates to treat a subject in need thereof, and any combination thereof.

### EXAMPLES

Some aspects of the embodiments discussed above are disclosed in further detail in the following examples, which are not in any way intended to limit the scope of the present disclosure.

### Example 1

### CDRexAb: Antibody Small-Molecule Conjugates with Computationally-Designed Target-Binding Synergy

Antibody-drug conjugates (ADCs), or chimeric modalities in general, combine the advantages and offset the flaws of the constituent parts to achieve a broader target space than traditional approaches of pharmaceutical development. As disclosed herein, full atomic simulation capability of computational protein design was used to define a new class of molecular recognition agents: CDR-extended antibodies, abbreviated as CDRexAbs. A CDRexAb incorporates a small-molecule binding event into de novo designed antibody/target interactions, creating antibody small-molecule conjugates that bind tighter against the target of the small molecule than the small molecule itself.

Monomeric streptavidin/biotin pairs at either a nanomolar or micromolar-level affinity. In this example, nanobody-biotin conjugates designed using the methods disclosed herein exhibited >20-fold affinity improvement against the protein targets, with step-wise optimization of binding kinetics and the overall stability. This example demonstrated that the design workflow/methods disclosed herein can be used to improve small-molecule based therapeutics.

### Computationally-Designed Nanobody Small-Molecule Conjugation Creates Tighter Binders Against the Small-Molecule Target Protein

It was determined whether computationally-determined nanobody sequences and their designed conjugation to a small molecule can exhibit an enhanced binding affinity to the small-molecule target. Designing the antibody components of synergistically-binding ADCs can involve creating new antibody/target interface, which is challenging, largely because of the difficulty in predicting the global minimum conformation of antibody CDR loops against a targeted surface, while accurately modeling long structured loops remains a challenge in general. To restrict unpredicted CDR conformations that could lead to non-binding designs, an approach similar to the anchored-design methods was adopted. Anchored-design creates new protein-protein interfaces by first identifying hotspot residues that favorably interact with the target, then designing protein scaffolds to stabilize the anchoring hotspots. For synergistically-binding ADCs, the conjugated small molecule can be viewed as a hotspot “residue” that interacts with the target protein. Therefore, to create co-targeting ADCs, the drug can be designed as an anchoring non-natural CDR residue that is strengthened by additional CDR-target interactions, integrating the drug-target interaction into the antibody-target binding event, and forcing the CDRs to more likely adopt the designed conformation.

An exemplary non-limiting design strategy disclosed herein can include the following steps: predicting the optimal CDR binding poses against the target surface, and searching for the ideal conjugation strategy that accommodates both the optimized CDR pose and the target-small molecule interaction. For demonstration purpose, monomeric streptavidin was chosen in this example as a model target and biotin as our model small molecule. Streptavidin-biotin interactions have been extensively studied with high-resolution crystal structures available for reliable design. Tetrameric streptavidin binds to biotin with almost the highest-possible affinity, but multiple monomeric streptavidin constructs were reported with >10⁵-fold reduced biotin-binding affinity. So as a model system, monomeric streptavidin-biotin interaction pairs not only can provide room for affinity improvement, but also have a known affinity upper limit, thus ideal for method development.

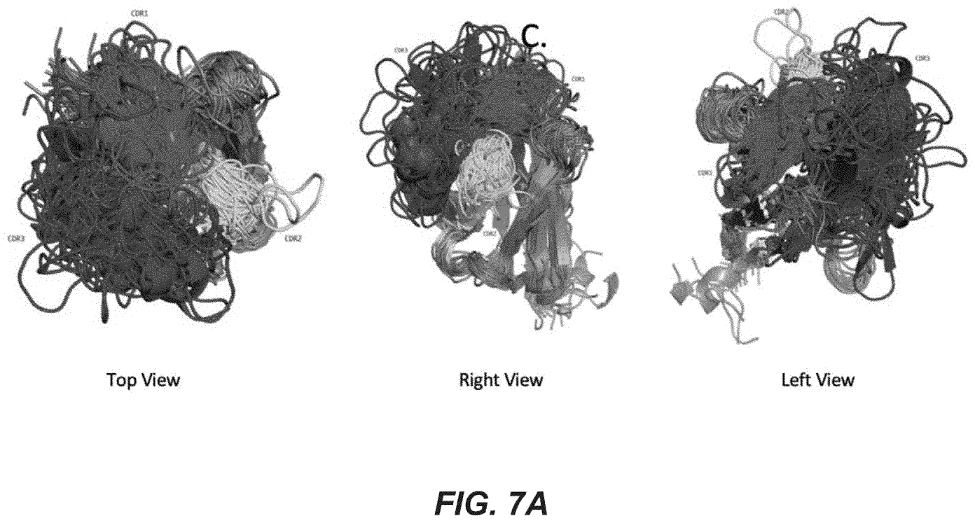

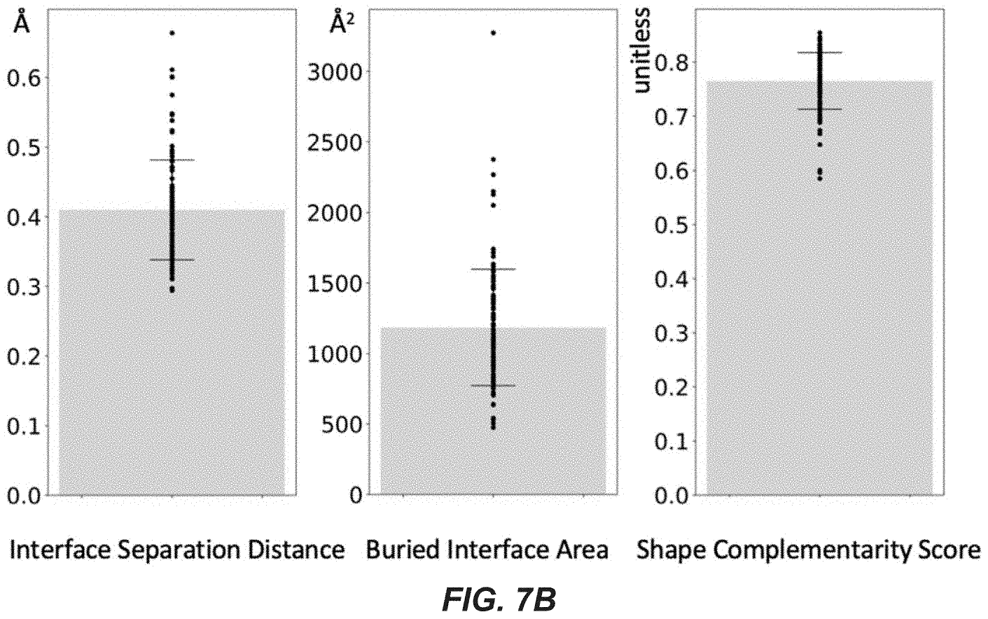

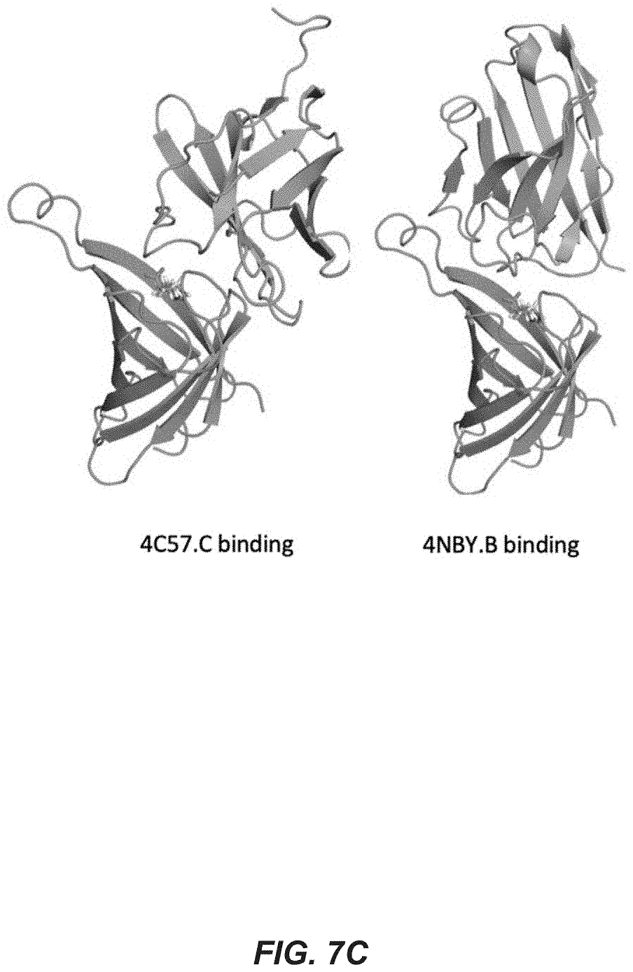

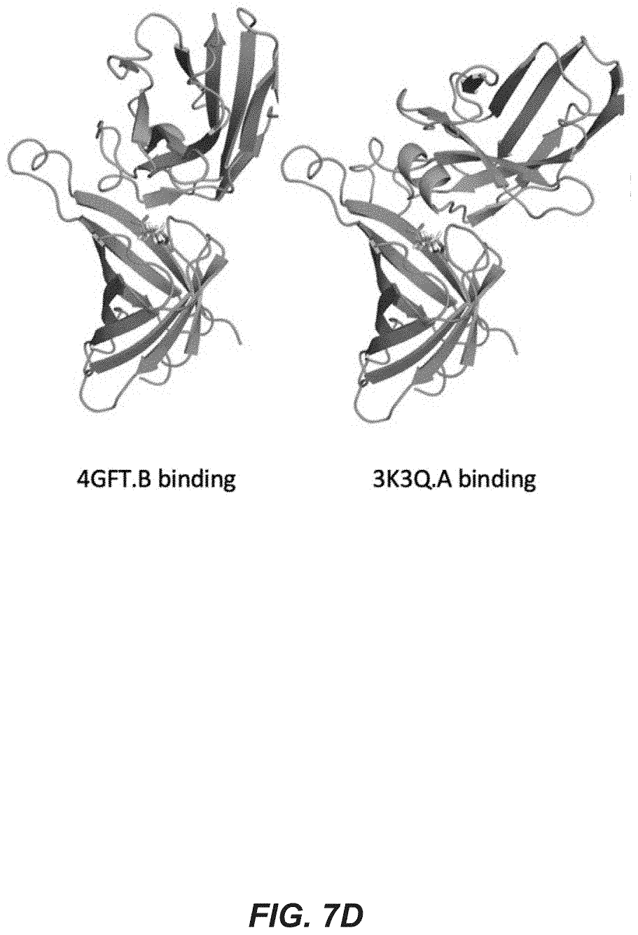

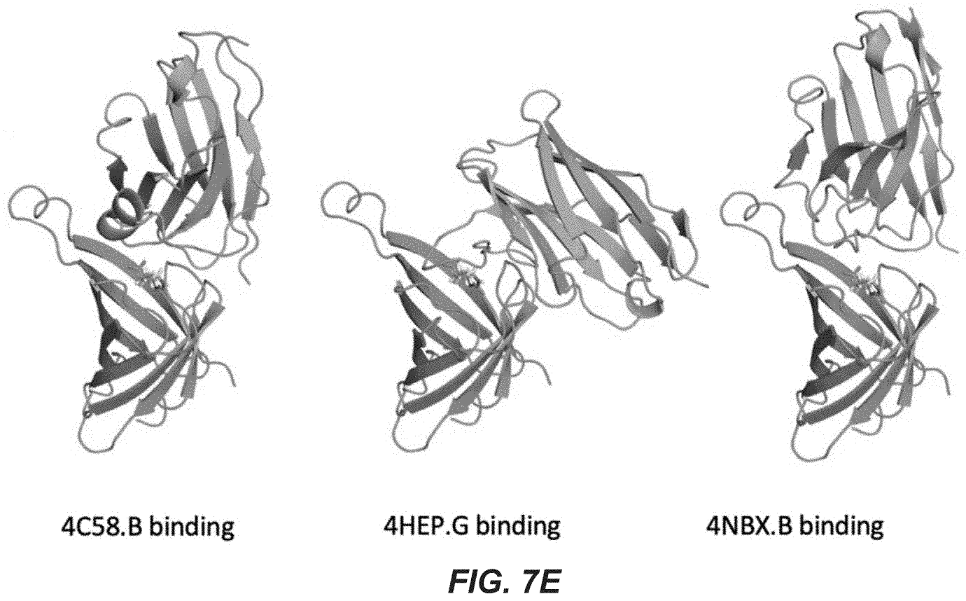

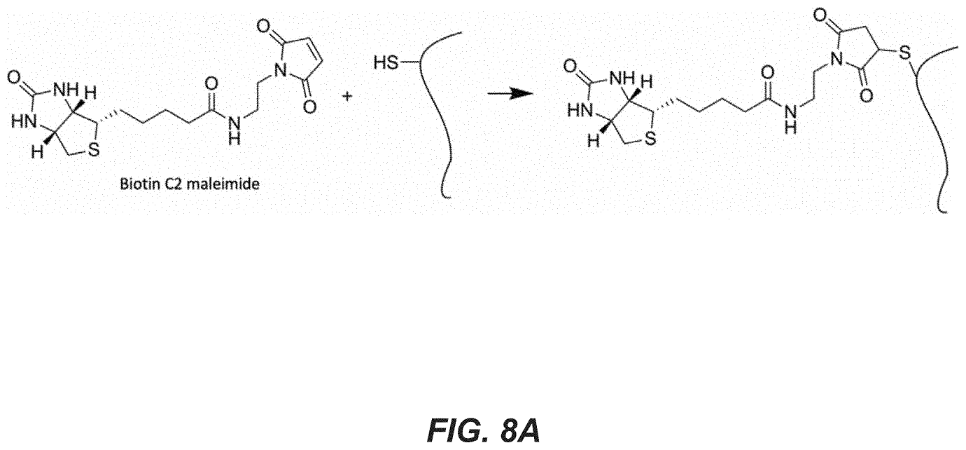

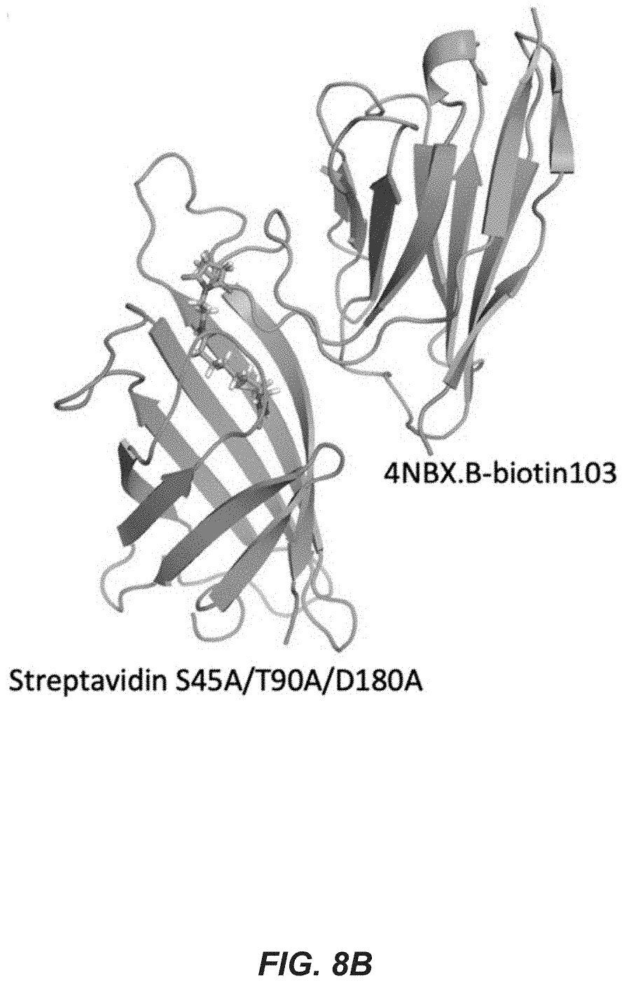

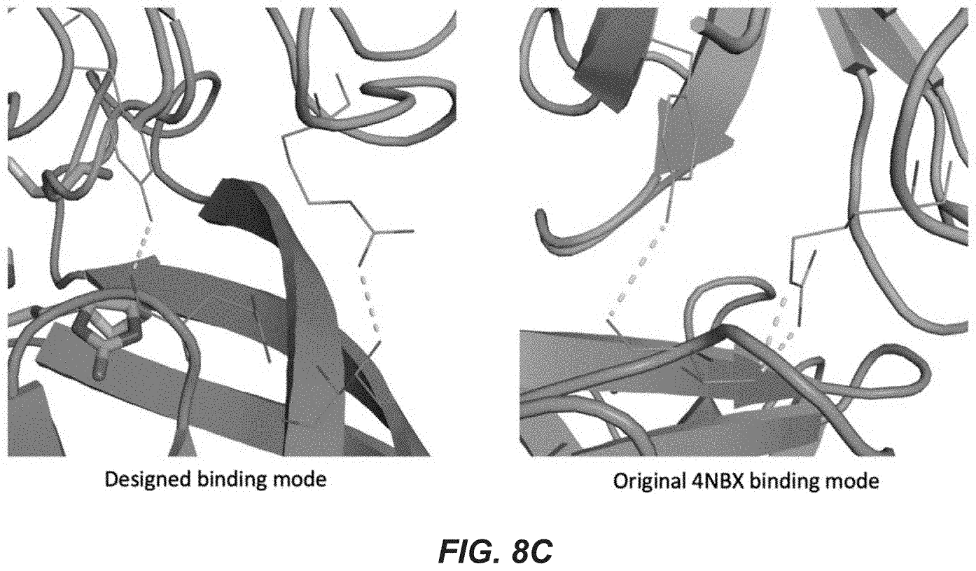

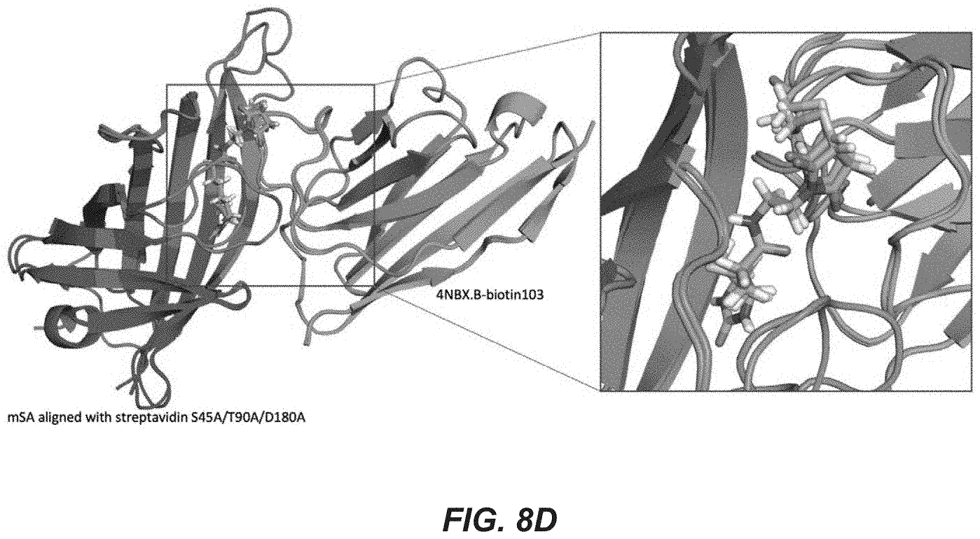

To search the optimal CDR binding conformations, a starting nanobody scaffold was first docked onto a monomeric core-streptavidin structure (SEQ ID NO:15, Table 4) with computationally-modeled sidechain replacements S45A/T90A/D180A (SEQ ID NO:16, Table 4), which were reported to monomerize streptavidin and reduce the biotin-binding affinity to 1.7 μM, and then performed loop-modeling on docked poses to attempt optimizing CDR conformations against the target surface. Most of the top loop modeling solutions were not representative of naturally occurring interactions. To sample realistic CDR structures, only previously-observed nanobody CDR binding conformations were searched around. Nanobody structures with diverse target-binding CDR conformations from PDB were curated, and individually docked them onto the target surface (FIG. 7A). 2310 docked poses were generated and filtered to potentially identify most realizable binding conformations, returning 7 final binding poses (FIG. 7C-FIG. 7E). Optimal conjugation strategy was then searched on the finalized poses. Biotin was conjugated onto nanobody CDRs by the cysteine-maleimide chemistry, which is a commonly used conjugation method in ADCs (FIG. 8A). Biotin C2 maleimide was chosen to be the conjugation reagent. Optimal nanobody scaffolds and conjugation sites were determined by computationally screening a rotamer library of the cysteine-conjugated side chain on the finalized nanobody-streptavidin poses. The top-ranked conjugation plan was amino acid site 103 of the nanobody scaffold 4NBX.B (chain B of PDB structure 4NBX, SEQ ID NO:1, Table 4), which originally binds to a target unrelated to any streptavidin construct. From the relaxed structure of the conjugate named as “4NBX.B-biotin103” in complex with monomeric streptavidin, Y112 and R27 of 4NBX.B are predicted to form hydrogen bonds with the target surface, whereas in the original PDB structure, these two residues also participated in H-bond formation, indicating that the designed pose is closely related to the natural binding mode of 4NBX.B, and potentially stabilized by specific CDR-target interactions upon biotin anchoring (FIG. 8B-FIG. 8C).

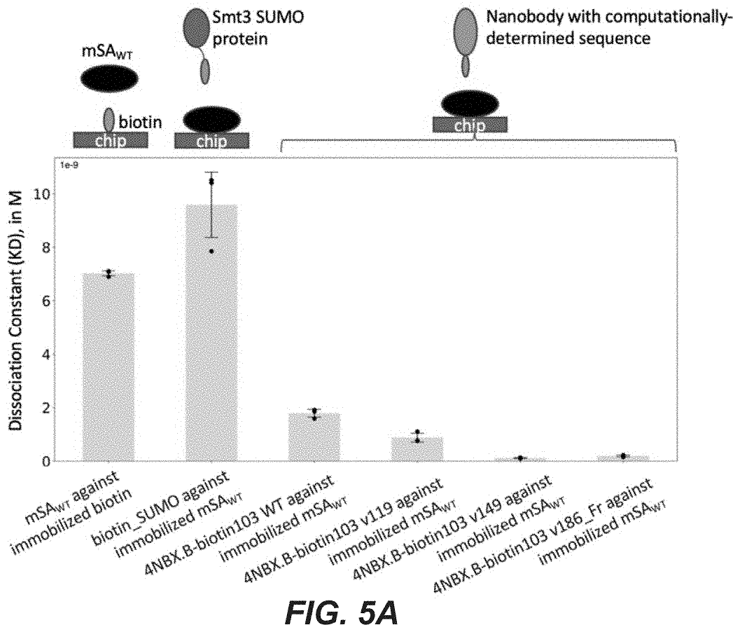

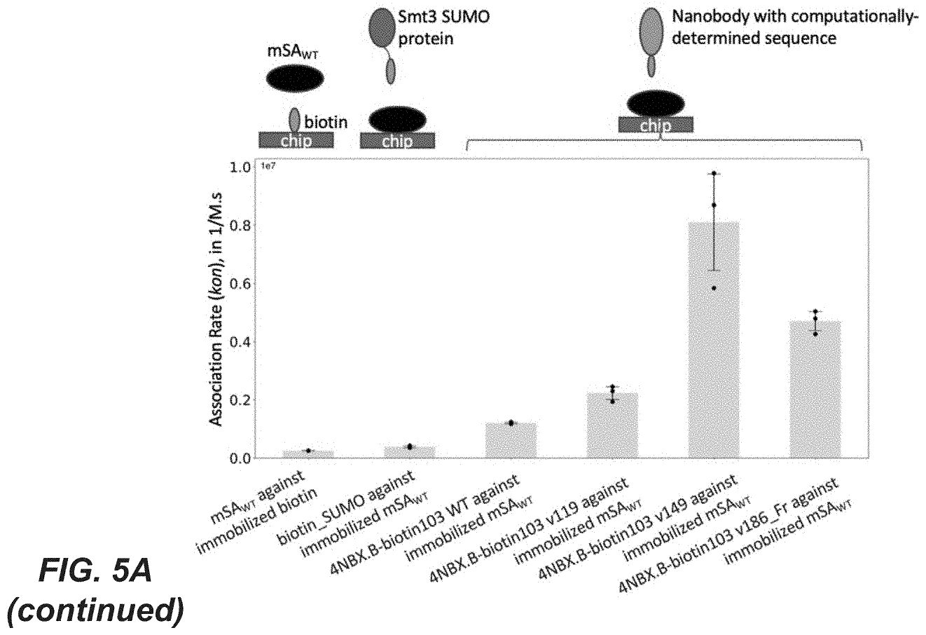

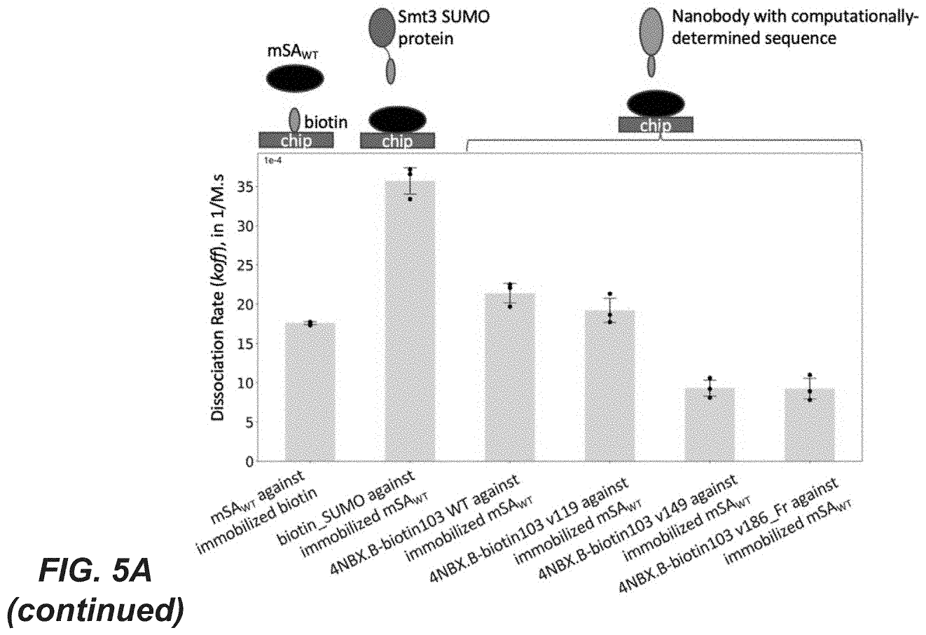

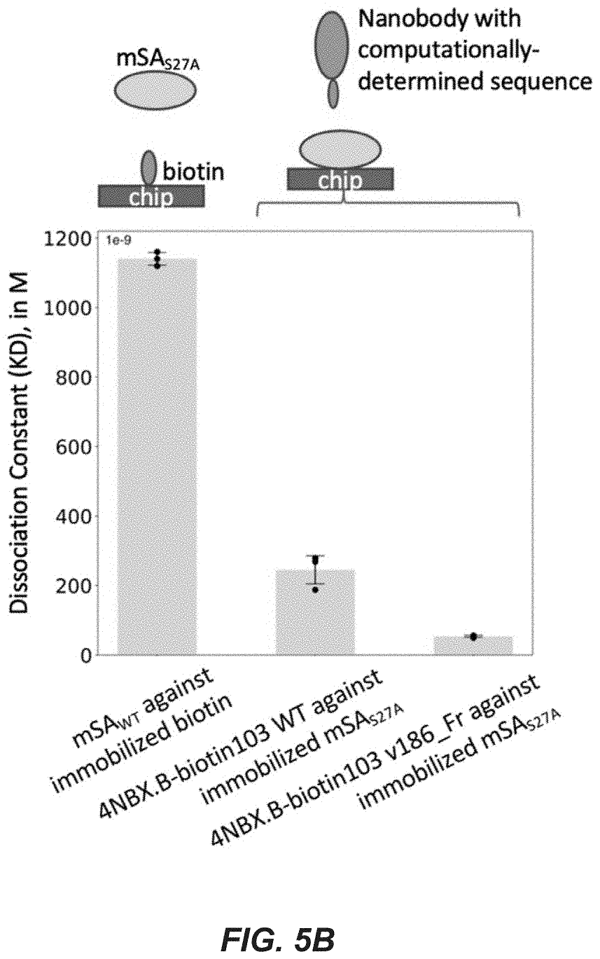

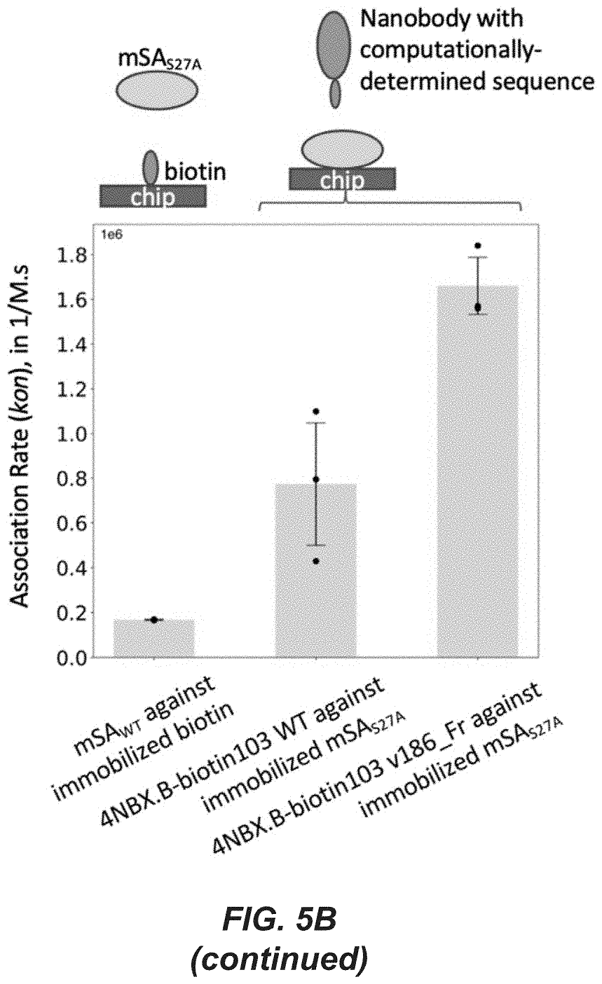

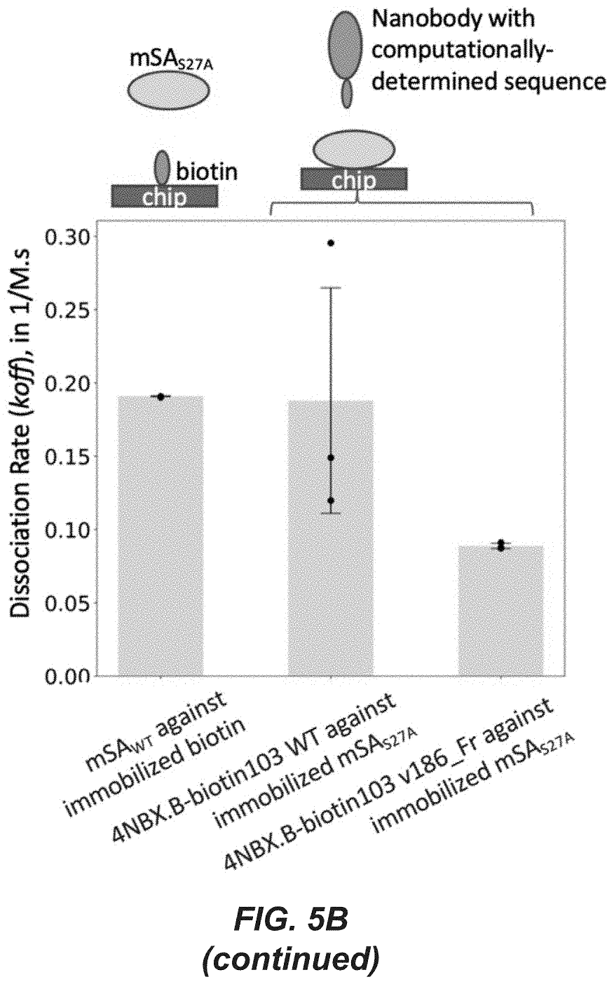

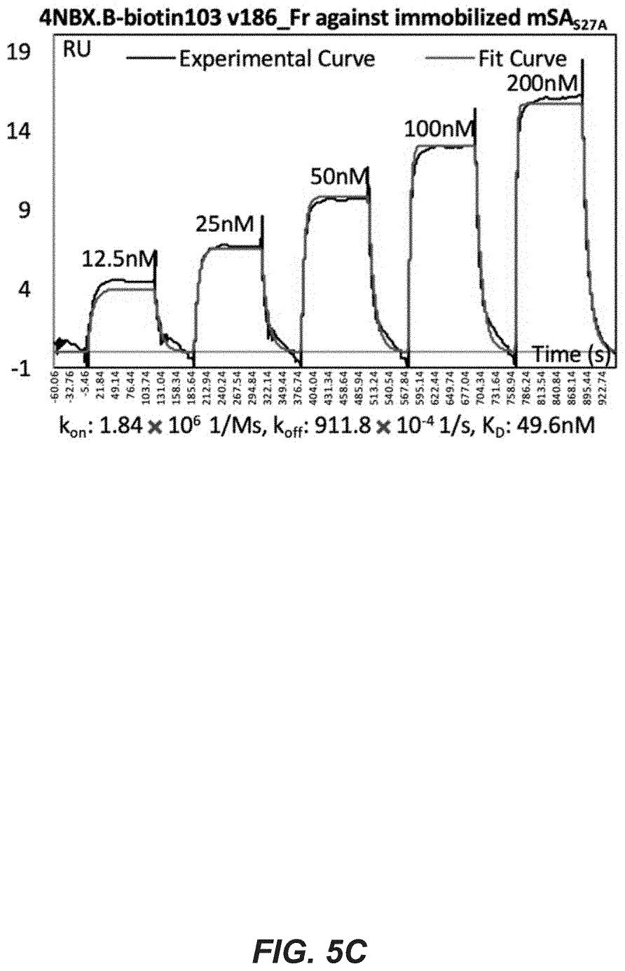

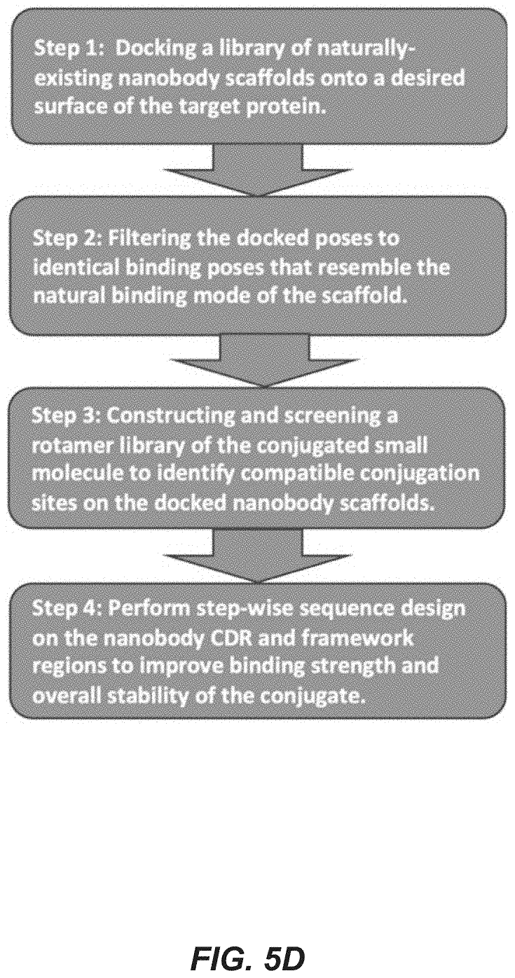

4NBX.B with site 103 mutated to cysteine was then synthesized (SEQ ID NO:2, Table 4), and conjugation with biotin C2 maleimide was performed. It was attempted to purify and refold the S45A/T90A/D128A mutant of core-streptavidin to perform binding measurement, but the resulted construct was unstable, as most proteins precipitated during refolding, and the refolded materials also quickly precipitated. Therefore, another previously-reported monomeric streptavidin construct, mSA (SEQ ID NO:17, Table 4), was aligned onto the triple-mutation streptavidin model that mSA is homologous to (sequence pairwise identity: 57%, structure RMSD: 0.5 Å), and relaxed 4NBX.B-biotin103 against mSA (FIG. 8D). The 4NBX.B-biotin103/mSA model preserved the rotamer configuration of conjugated biotin against the triple-mutation streptavidin, and H bonds contributed by Y112 and R27 were also recapitulated, and potentially participated in a broader predicted H-bond network that incorporated biotin/mSA interactions, suggesting that 4NBX.B-biotin103 may bind to mSA with the designed beneficial synergy (FIG. 8D, FIG. 1B-FIG. 1C). Indeed, surface plasmon resonance (SPR) binding experiments confirmed that under 25° C. 4NBX.B-biotin103 binds to immobilized mSA with a K_{D} of 1.8±0.1 nM, and mSA binds to immobilized biotin with a K_{D} of 7.0±0.1 nM, indicating a moderate 4-fold affinity improvement that is contributed by a higher kₒₙ(FIG. 1D-FIG. 1E, FIG. 5A). Wildtype 4NBX.B did not show binding signal to mSA at concentrations up to 100 nM, indicating that the 4NBX.B-biotin103 conjugate binds to the targeted biotin binding pocket (FIG. 1E top panel). The SPR-measured biotin/mSA affinity is similar to previously-published fluorescence polarization spectroscopy data, which is 2.8±0.5 nM under 4° C. and 5.5±0.2 nM under 37° C. However, because the data fitting quality of the mSA/biotin binding curves is lower than the 4NBX.B-biotin103 binding curves, to confirm the estimated mSA/biotin affinity, an alternative estimation was performed by binding immobilized mSA to N-terminal biotinylated Smt3 SUMO protein. Smt3 SUMO protein has an unstructured N-terminus and it was hypothesized this would minimize the interaction between the protein components. A similar K_{D} is estimated with high data-fitting quality, indicating that the measured biotin/mSA affinity is an accurate SPR estimation (FIG. 1D bottom panel).

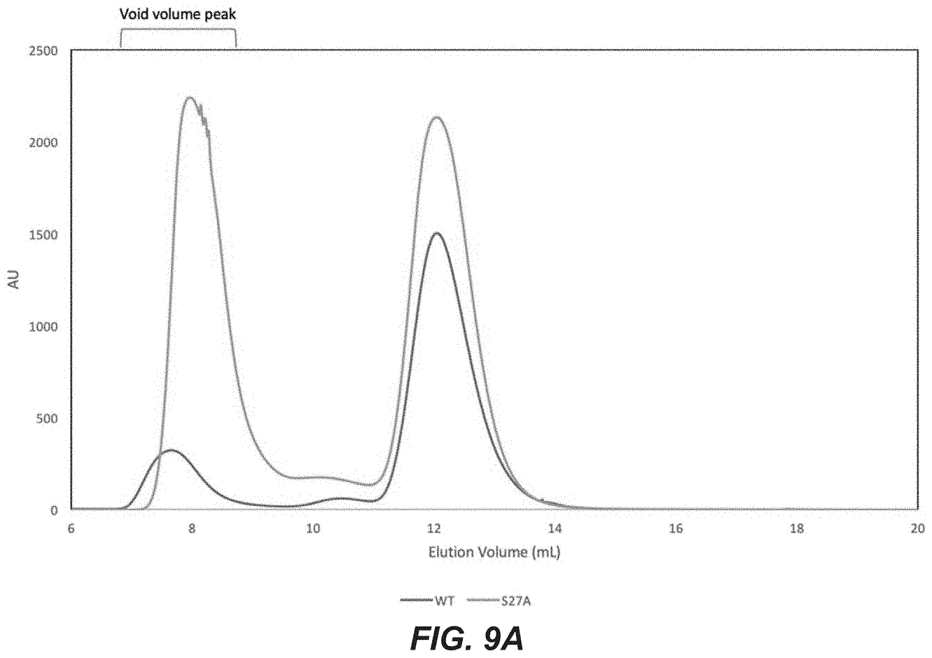

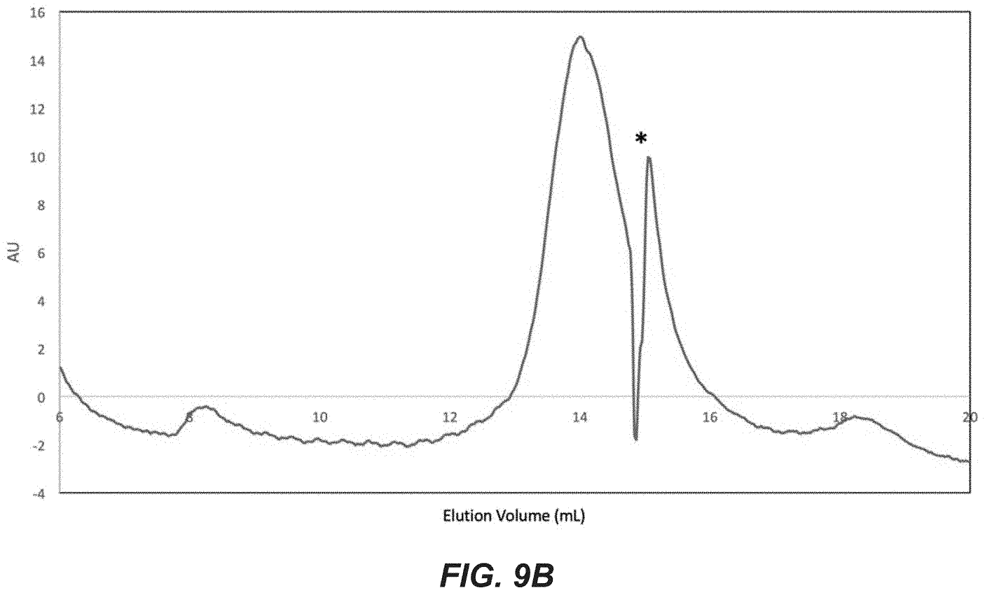

To determine whether computationally-designed nanobody conjugation shows improved affinity with weakly-binding small molecules, a single mutation S27A on mSA was created (SEQ ID NO:18, Table 4), whose counterpart S45A in wild type streptavidin reduces biotin-binding strength and was predicted by molecular dynamics (MD) simulation to minimally affect the overall structure. On size-exclusion chromatography (SEC), mSA_{S27A} is eluted at the same time as mSA_{WT} (FIG. 9A). SPR estimated that mSA_{S27A} binds to biotin with a K_{D} of 1.14±0.02 μM, while 4NBX.B-biotin103 binds to mSA_{S27A} with a K_{D} of 245±41 nM, indicating a similarly-moderate 5-fold improvement (FIG. 1F, FIG. 5B). Together, the above results showed that based on the sole structural information of a small molecule-target interaction, nanobody conjugation to the small molecule can be designed entirely by computational methods to exhibit an affinity-enhancing synergistic binding effect.

### Sequence Design Further Improves the Binding Affinity and Kinetics for Computationally Designed Conjugates

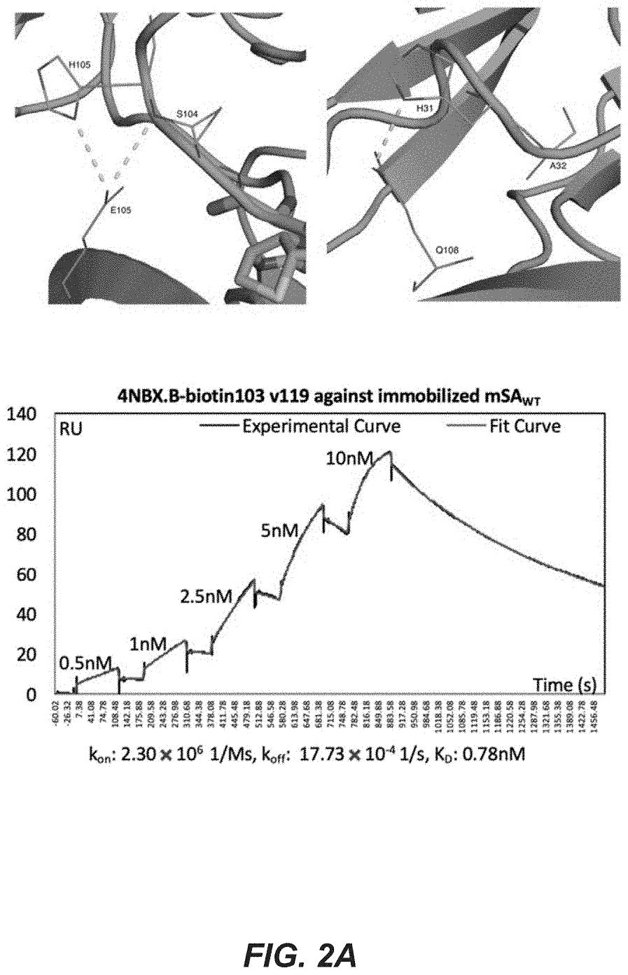

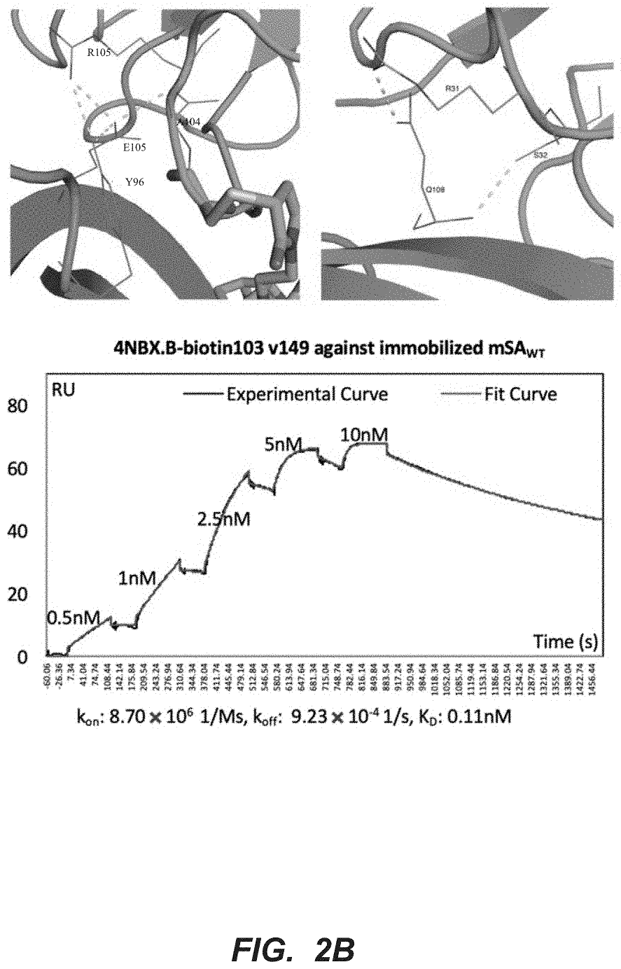

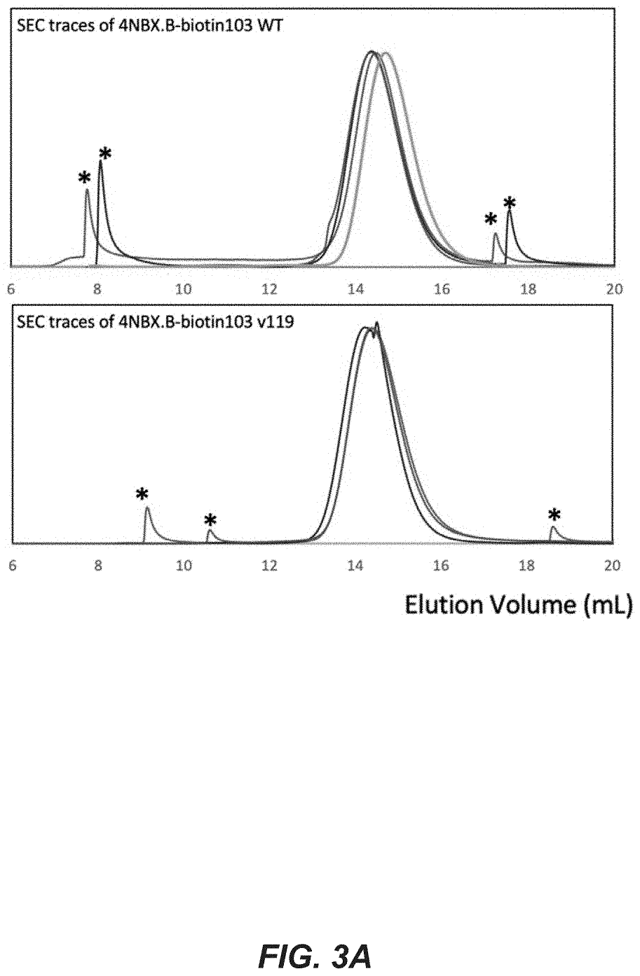

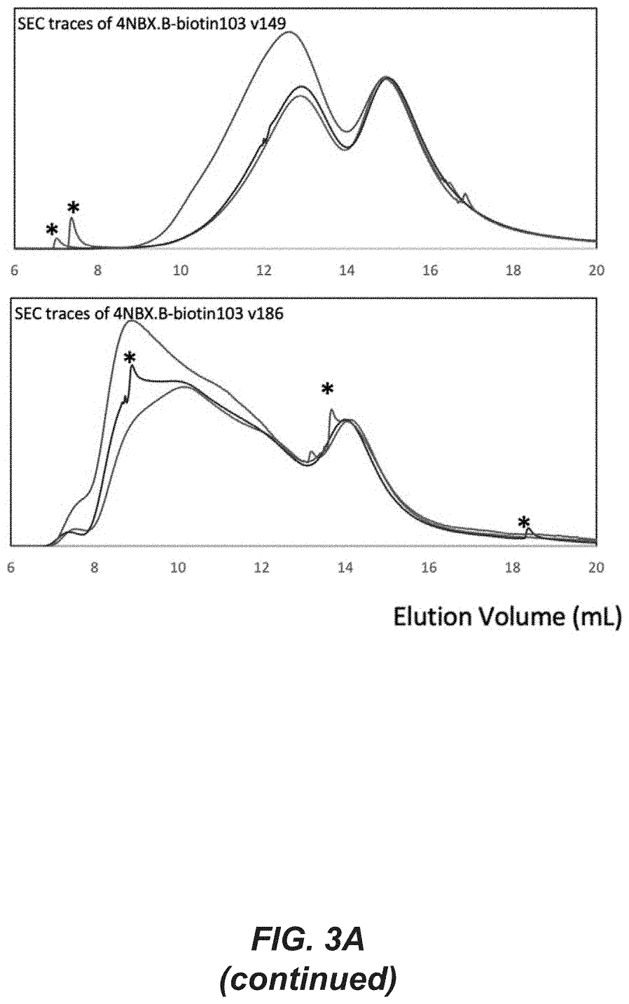

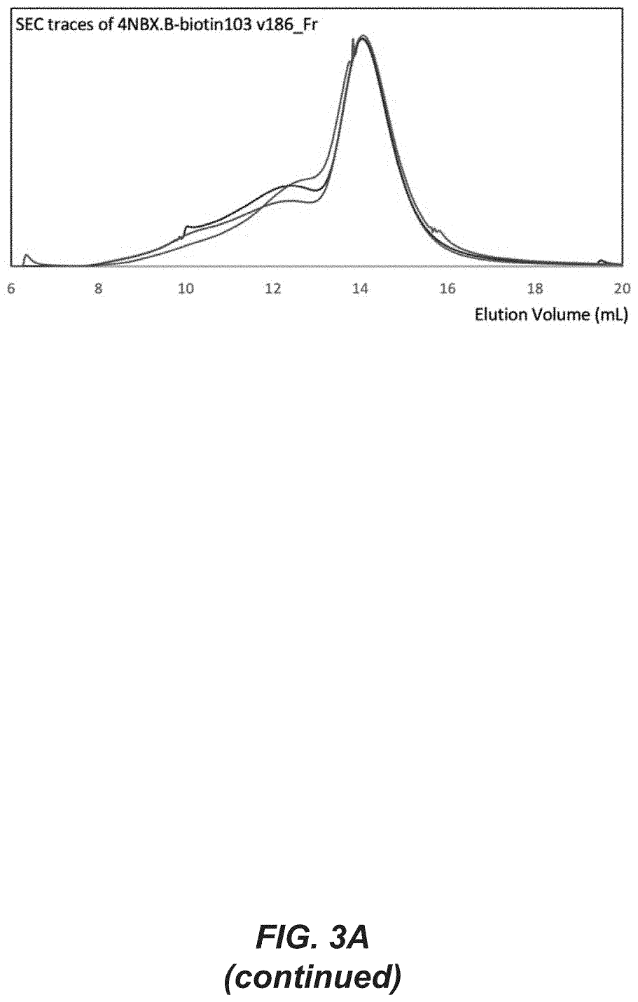

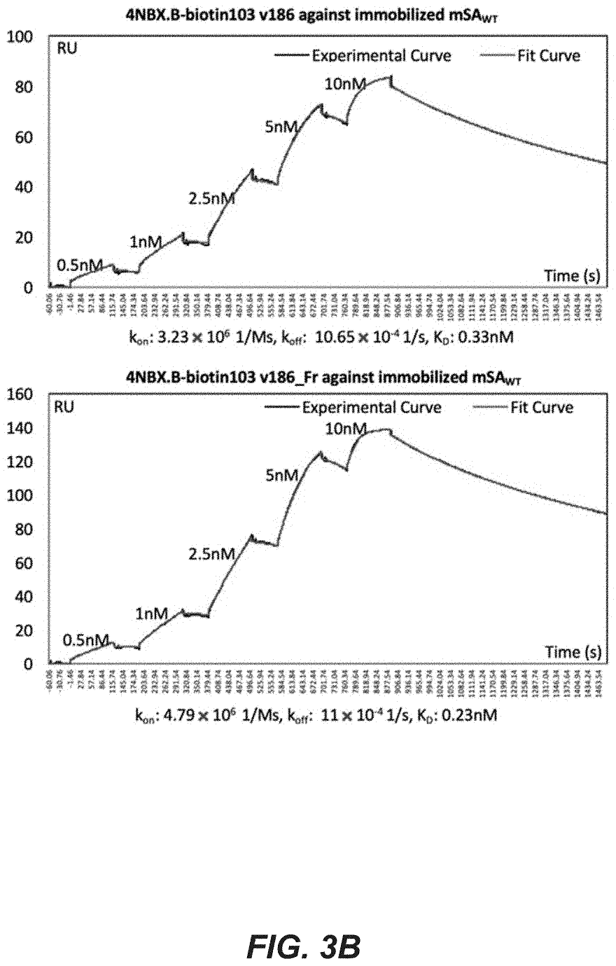

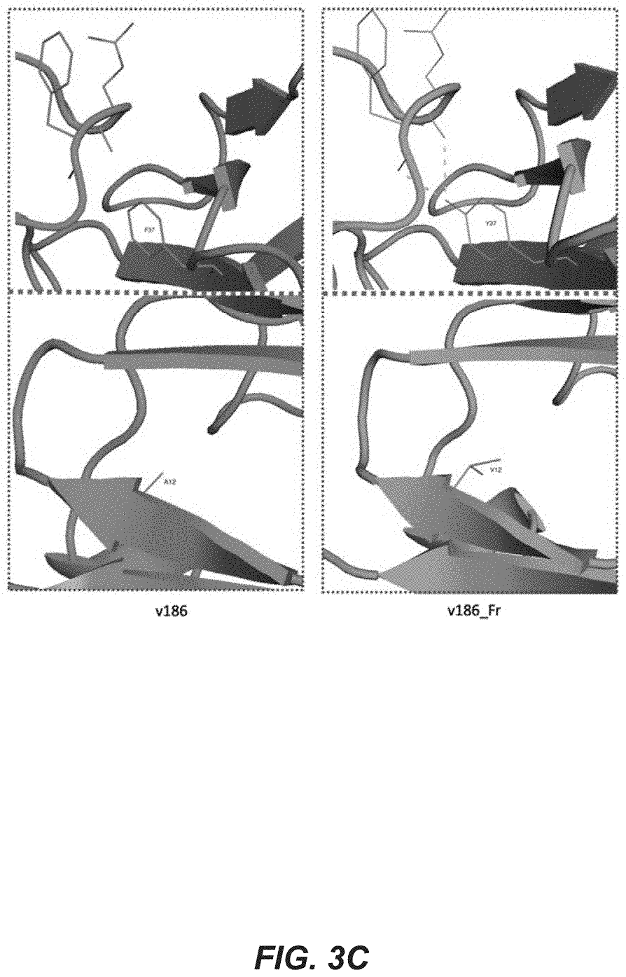

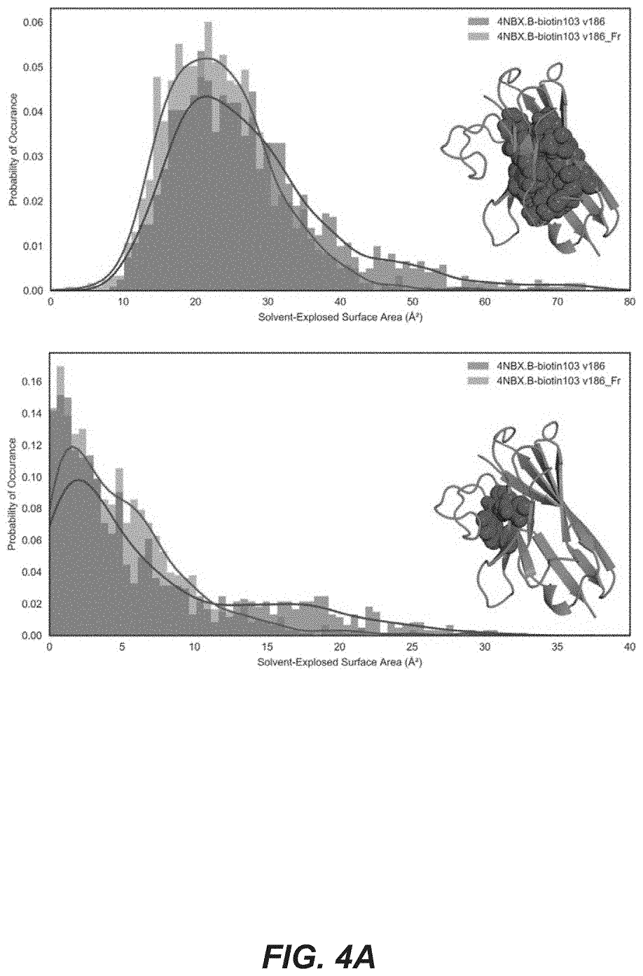

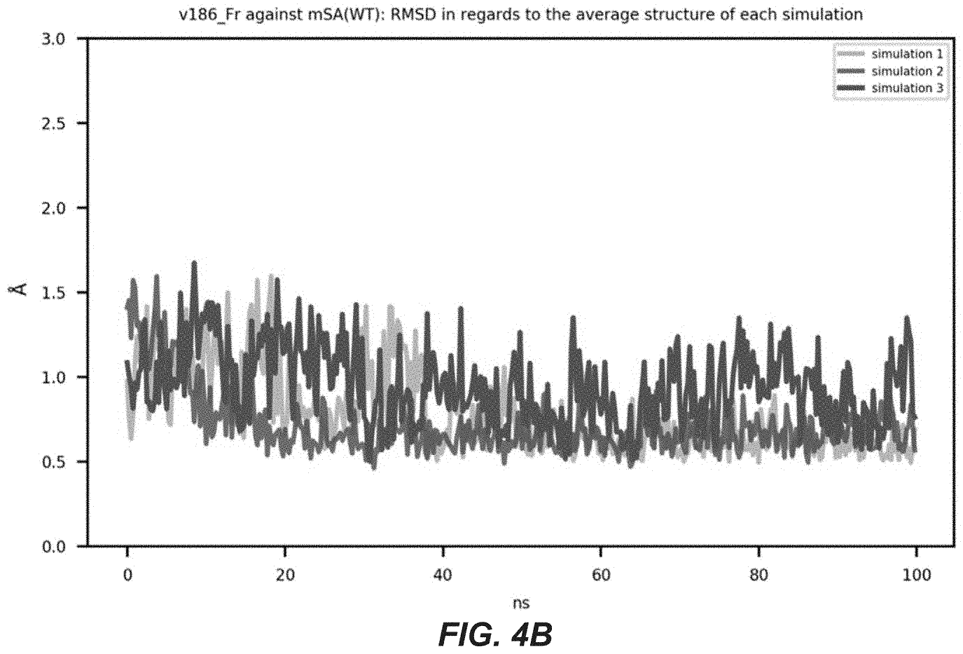

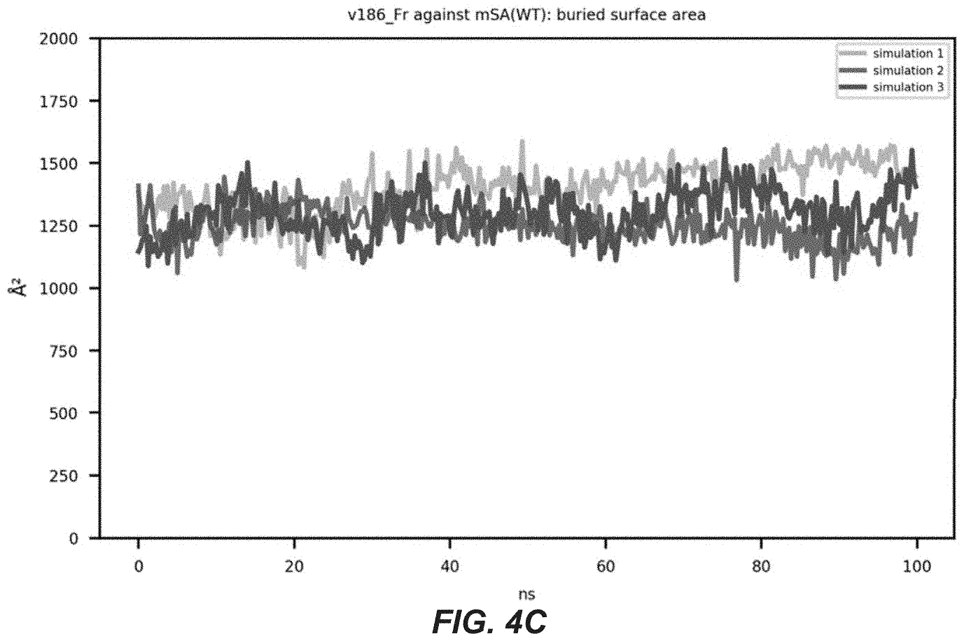

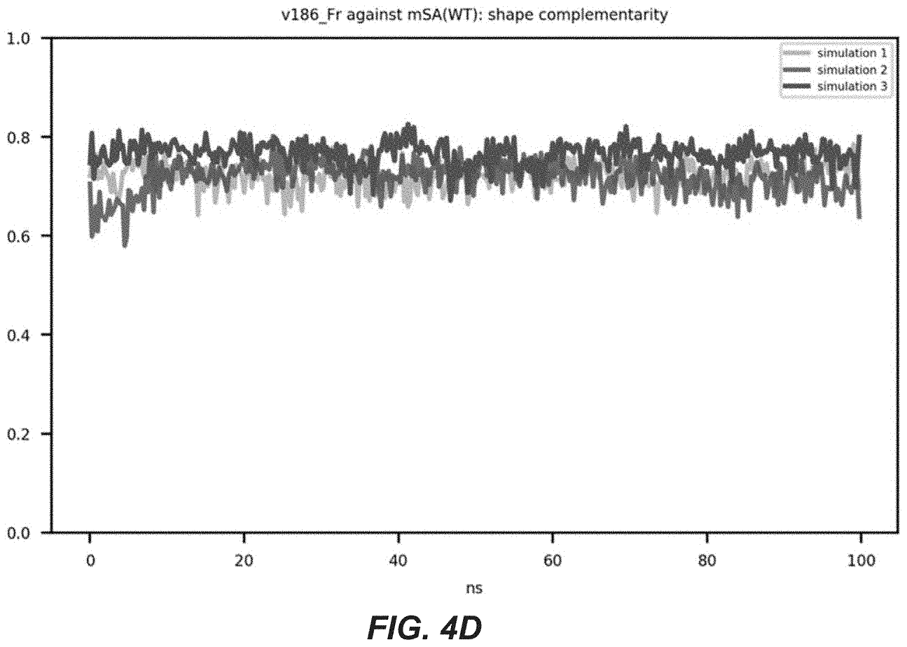

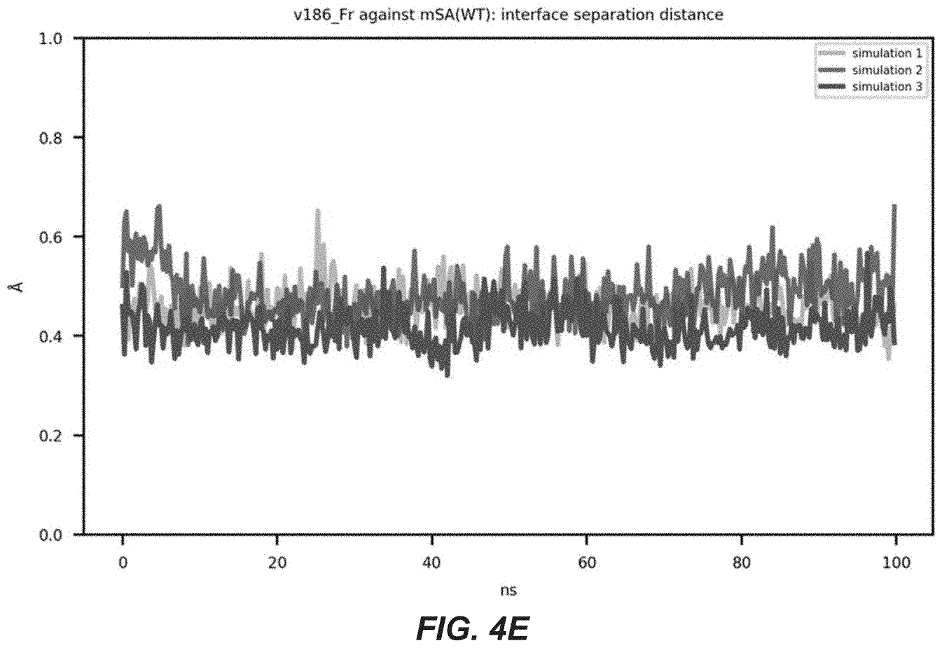

Next, sequence design on the CDR loops of 4NBX.B-biotin103 was performed to improve its binding affinity against mSA and further validate the accuracy of the modeled binding pose. Each CDR amino acid site was analyzed in silico for its favorability of accepting mutations, and combinatorial designs were performed on the mutable sites. Four combinations with different site-selection biases were tested in parallel, and the residue choices for each site were decided according to a published study on the sequence diversity of nanobody CDR loops. Analysis of design outputs revealed that the design with sites 31, 32, 104, and 105 most frequently returned sequences that were likely to form additional H-bonds with mSA and were also energetically stable. The top-ranked variant by energy, v119 with CDR1 mutations M31H/D32A and CDR3 mutations N104S/W105H, was predicted to form new H-bonds with residue Q108 of mSA by H31 and with E105 of mSA by H105 (Table 1, FIG. 2A, SEQ ID NO:3, Table 4). The D32A mutation also eliminates a buried charged residue in the designed interface, which would be a severe energetic penalty. SPR measured the K_{D} of 4NBX.B-biotin103 v119 against mSA_{WT} to be 0.9±0.2 nM, indicating a ˜2-fold improvement from 4NBX.B-biotin103 WT (FIG. 2A, FIG. 5A). The K_{D} improvement was again mainly contributed by kₒₙ increase, and the observed k_{off} values were only minimally different (FIG. 2A, FIG. 5A). To obtain a variant that would more significantly reduce the k_{off}, variant v149 was picked that has the highest number of predicted H-bond formation from the top 20 output sequences. 4NBX.B-biotin103 v149 has mutations M31R/D32S/N104A/W105R that were predicted to form more extensive H-bonds with Y96, E15, and Q108 of mSA, with a potential salt bridge between the nanobody R105 and mSA E105 (FIG. 2B, SEQ ID NO:4, Table 4). R-E interactions are frequently used by nanobodies, further validating this designed interaction. Indeed, compared to v119, SPR measured a ˜0.2-fold slower k_{off} and a ˜4-fold faster kₒₙ for v149, which together contribute to the K_{D} of 0.12±0.01 nM, indicating a >20-fold K_{D} improvement from biotin/mSA_{WT} affinity (FIG. 2B, FIG. 4A). However, according to SEC traces, v149 seemed to be very prone to aggregation, indicating protein instability (FIG. 3A).

TABLE 1
TOP 20 SEQUENCE OUTPUTS FROM CDR DESIGN OF
SITE 31, 32, 104, AND 105 ON 4NBX.B-BIOTIN103 WT.
(RANKED BY ENERGY SCORE)

| | Energy | | Mutations: chain name and |
|---|---|---|---|
| Ranking | Score | Note | accepted residue |
| 1 | −457.58628 | v119 | B_31H + B_32A + B_104S + B_105H |
| 2 | −456.82531 | | B_31N + B_32S + B_104S + B_105S |
| 3 | −456.56855 | | B_31N + B_32S + B_104S + B_105A |
| 4 | −456.56059 | | B_31R + B_32A + B_104D + B_105R |
| 5 | −456.50215 | | B_31R + B_32A + B_104S + B_105Y |
| 6 | −456.42944 | | B_31N + B_32A + B_104S + B_105S |
| 7 | −456.25964 | | B_31Q + B_32A + B_104S + B_105Y |
| 8 | −456.13421 | | B_31H + B_32S + B_104S + B_105V |
| 9 | −456.11428 | | B_31N + B_32A + B_104S + B_105R |
| 10 | −455.81205 | | B_31N + B_32A + B_104S + B_105A |
| 11 | −455.7737 | | B_31N + B_32S + B_104S + B_105D |
| 12 | −455.67248 | | B_31R + B_32A + B_104D + B_105H |
| 13 | −455.6018 | | B_31Q + B_32A + B_104A + B_105R |
| 14 | −455.26629 | | B_31R + B_104S + B_105H |
| 15 | −455.13137 | | B_31H + B_32S + B_104D + B_105S |
| 16 | −455.10018 | | B_31N + B_32S + B_104A + B_105S |
| 17 | −455.05911 | v149 | B_31R + B_32S + B_104A + B_105R |
| 18 | −454.97734 | | B_31R + B_32S + B_104S + B_105Y |
| 19 | −454.93878 | | B_31K + B_32S + B_104A + B_105A |
| 20 | −454.90643 | | B_31Q + B_32S + B_104A + B_105A |

### Sequence Design Further Improves the Binding Affinity and Kinetics for Computationally Designed Conjugates

On SEC, both 4NBX.B-biotin 103 WT and v119 showed single peaks eluted roughly at the same time as wild type 4NBX.B nanobody, indicating stabilized monomer foldedness (FIG. 3A). The reduced monomer stability of v149 agrees with its predicted lower energy score than v119 (Table 1). Only four residues were designed to improve the stability of v149, further CDR designs were developed for the biotin103 side chain and the four H-bond contributing mutations, thus stabilizing the loop and overall structure. Two additional rounds of CDR residue mutability analysis were performed followed by in-parallel combinatorial designs on v149 until no further CDR mutations were predicted to be energetically favorable. Mutations accumulated in previous rounds of design were kept intact in subsequent rounds. In top 20 sequences ranked by energy score of both rounds of design, no additional H-bond was predicted to form with mSA, so the sequences with the best energy improvement were selected. The resulted variant, v186 (SEQ ID NO:5, Table 4), has 6 additional CDR mutations Y101L/R107F/R56T/Y106K/D108A/Y110S on top of v149, and was predicted to preserve the H-bonds contributed by v149 mutations. As expected, v186 binds to mSA_{WT} with very similar K_{D} as v149 (FIG. 3B top panel). However, SEC traces of v186 showed even worse aggregates formation than v149 (FIG. 3A).

MD simulations have been successfully applied to reveal the source of unexpected functional properties in designed proteins. To understand the flaws of the structure and inform next design strategy, MD simulation of 4NBX.B-biotin103 v186 in complex with mSA_{WT} was performed. From the simulation, it was noticed that the CDR3 loop that originally folded over the β-barrel framework region became gradually widened from the initial conformation, and eventually protruded away from the framework. The apparently destabilized loop-framework geometry suggests that the framework sequence is not fully compatible with the mutated CDR sequences, and needs to be adjusted. Therefore framework sequence design on v186 was performed, and the top-ranked variant v186_Fr (SEQ ID NO:6, Table 4) was predicted to form additional H-bonds with CDR3 residues through the F37Y mutation (FIG. 3C, Top). In addition, the A12V mutation increases the hydrophobic shielding of the β-barrel core (FIG. 3C, Bottom). It's also noted when the same framework sequence design was performed on v149, different from the v186 design, the A12V/F37Y mutations were predicted to be less energetically favorable than the parent v149, suggesting that the v186 mutations were a prerequisite for the A12V/F37Y mutations to be beneficial (Table 1-Table 2).

TABLE 2
TOP 20 SEQUENCE OUTPUTS FROM FRAMEWORK DESIGN
ON 4NBX.B-BIOTIN103 V186. (RANKED BY ENERGY SCORE)

| | Energy | | Mutations: chain name and |
|---|---|---|---|
| Ranking | Score | Note | accepted residue |
| 1 | −500.8748 | v186_Fr | B_12V + B_37Y |
| 2 | −500.3636 | | B_37Y + B_40A |
| 3 | −500.24131 | | B_5Q + B_12V + B_37Y |
| 4 | −500.10634 | | B_12V + B_35G + B_37Y |
| 5 | −499.9846 | | B_12V + B_37Y + B_40A |
| 6 | −499.90705 | | B_12V |
| 7 | −499.22799 | | B_12V + B_35G |
| 8 | −499.15484 | | B_5Q + B_12V + B_35G + B_37Y |
| 9 | −499.02353 | | B_12V + B_35G + B_37Y + B_40A |
| 10 | −498.98295 | | B_5Q + B_12V + B_35G + B_37Y + |
| | | | B_40A |
| 11 | −498.78612 | | B_37Y |
| 12 | −498.6384 | | B_12V + B_35G + B_40A |
| 13 | −498.54874 | | B_35G + B_37Y |
| 14 | −498.53802 | | B_12V + B_40A |
| 15 | −498.48109 | | B_5Q + B_12V + B_40A |
| 16 | −498.31249 | | B_5Q + B_37Y |
| 17 | −498.04085 | | WT |
| 18 | −497.75764 | | B_5Q + B_12V |
| 19 | −497.68241 | | B_5Q |
| 20 | −497.59715 | | B_5Q + B_35G + B_37Y |

4NBX.B-biotin103 v186_Fr showed significantly reduced aggregation on SEC. Collected fractions excluding the aggregates peak did not re-aggregate once rerun on SEC (FIG. 3A, FIG. 9B). SPR measured the K_{D} of v186_Fr to be 0.20±0.03 nM, which preserved the >20-fold K_{D} improvement from biotin/mSA_{WT} (FIG. 3B bottom panel, FIG. 5A). The kinetics profile of v186_Fr against mSA_{WT} was also similar to v149 (FIG. 3B bottom panel, FIG. 5A). When binding to mSA_{S27A}, v186_Fr exhibited K_{D} to be 54±3 nM, indicating a ˜20-fold K_{D} improvement contributed by both improved association rate and dissociate rate (FIG. 5B-FIG. 5C).

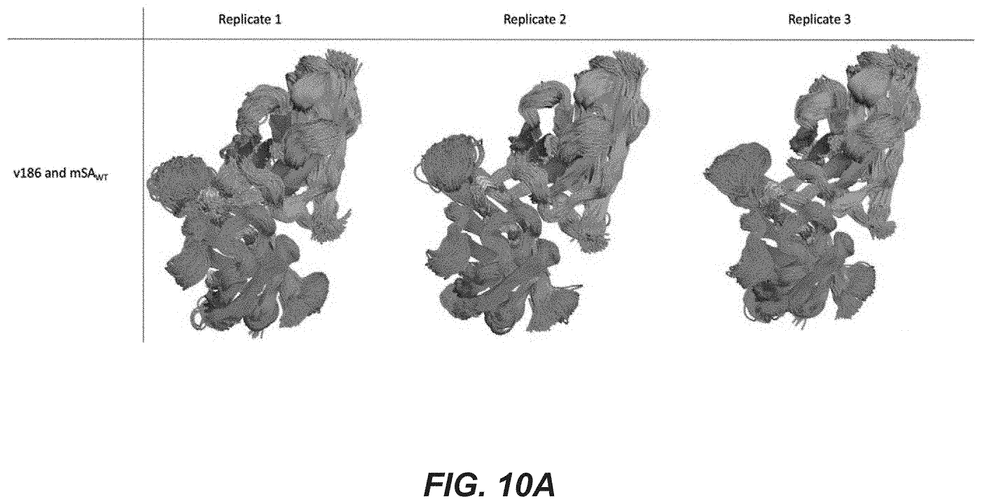

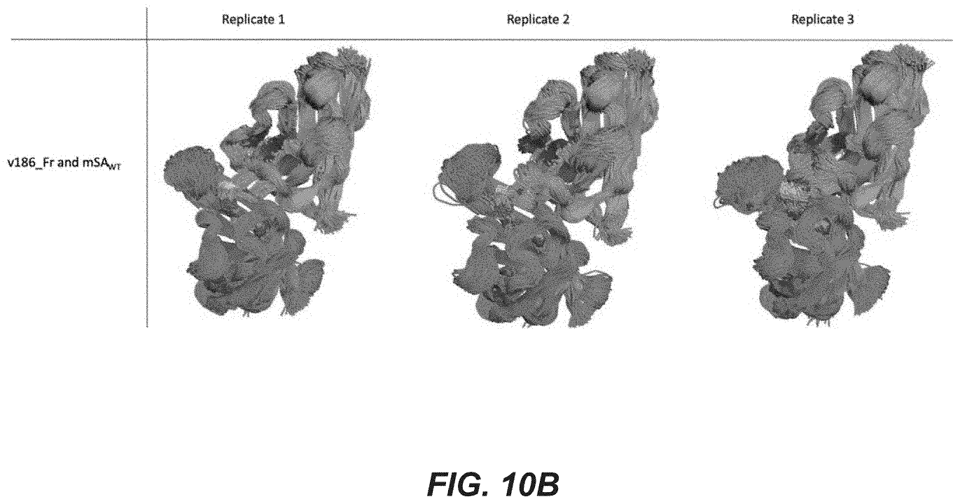

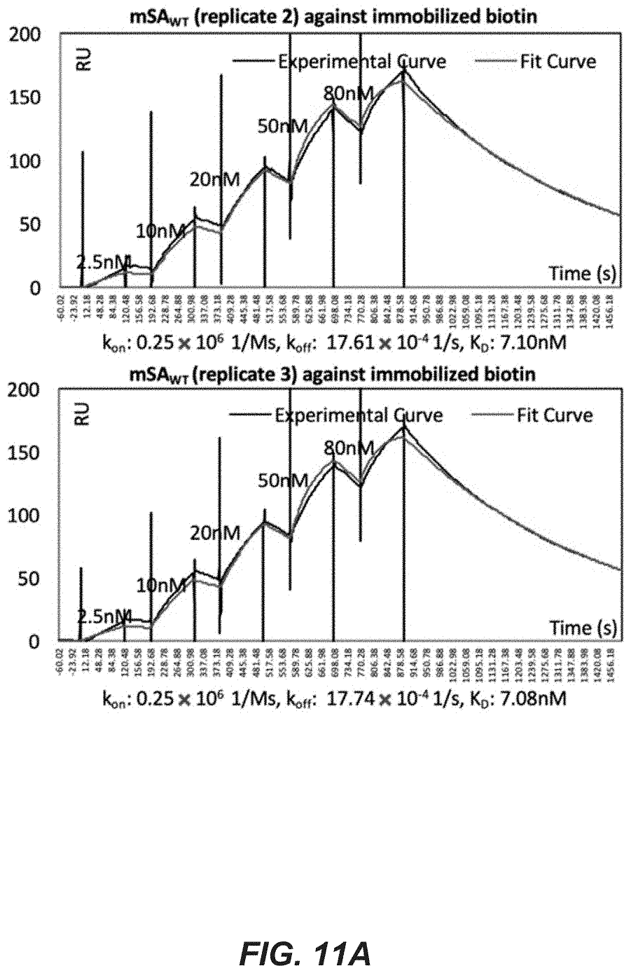

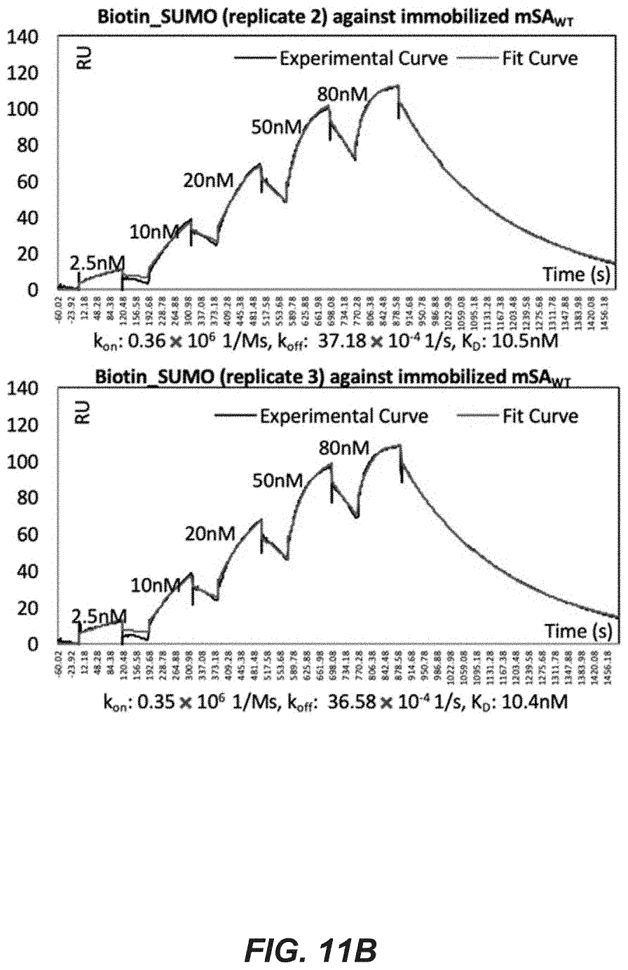

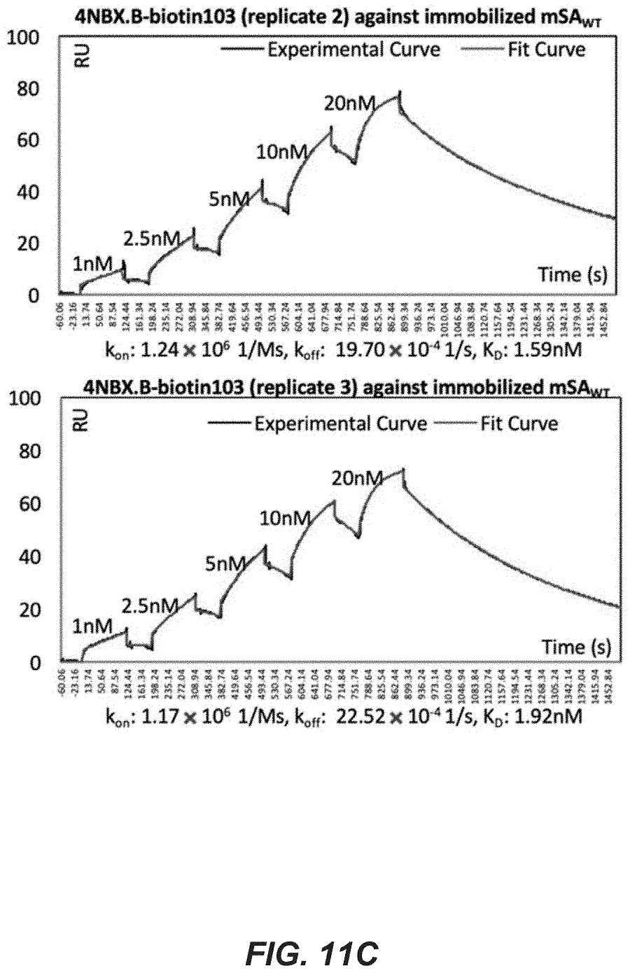

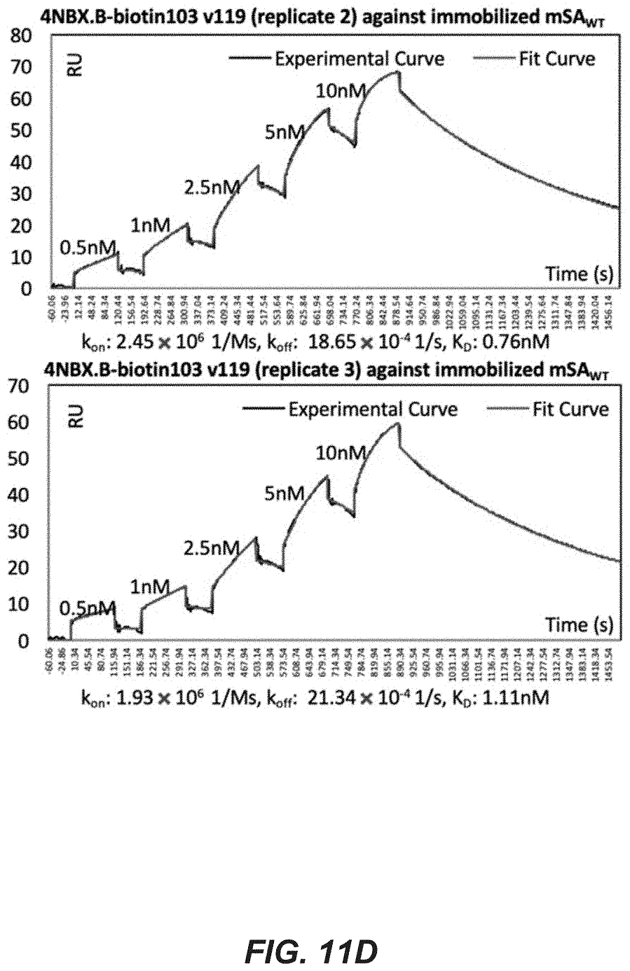

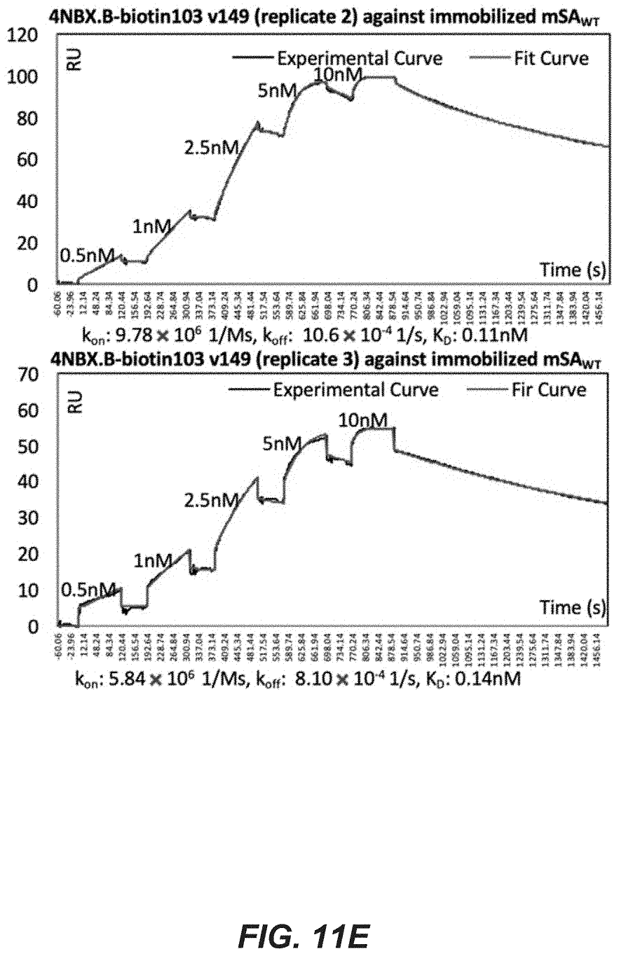

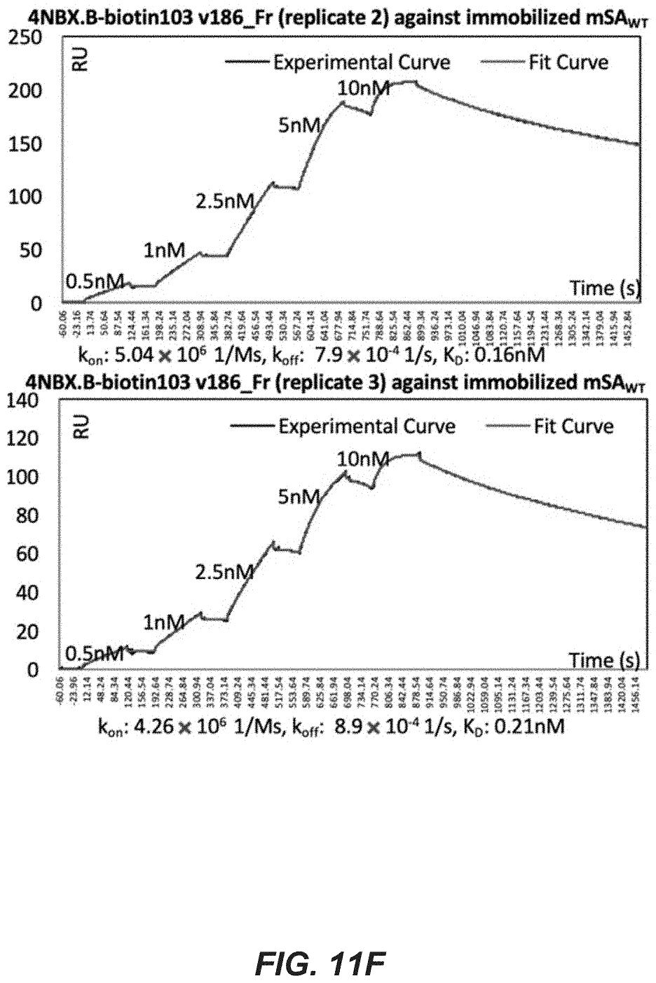

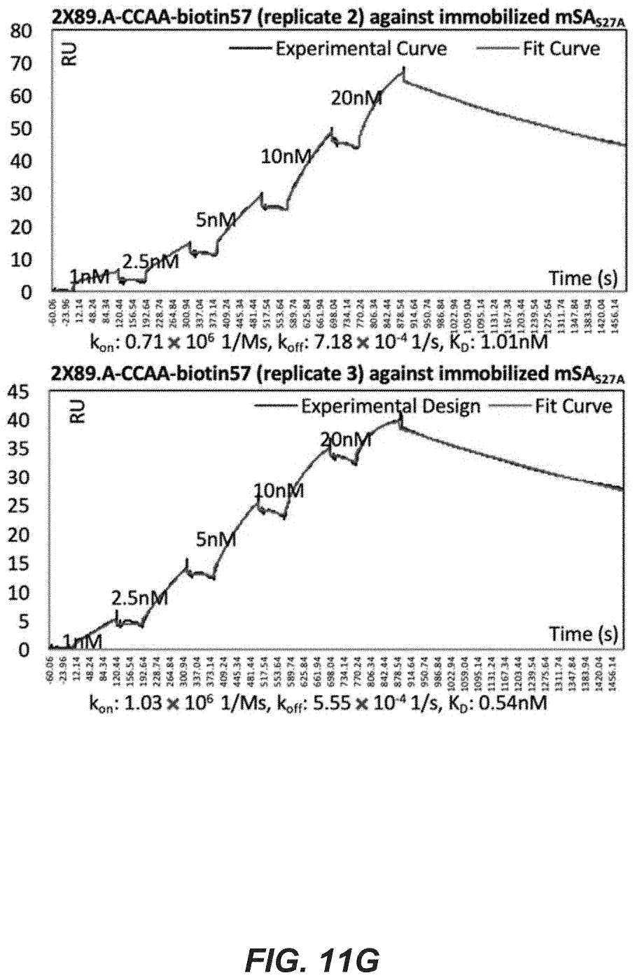

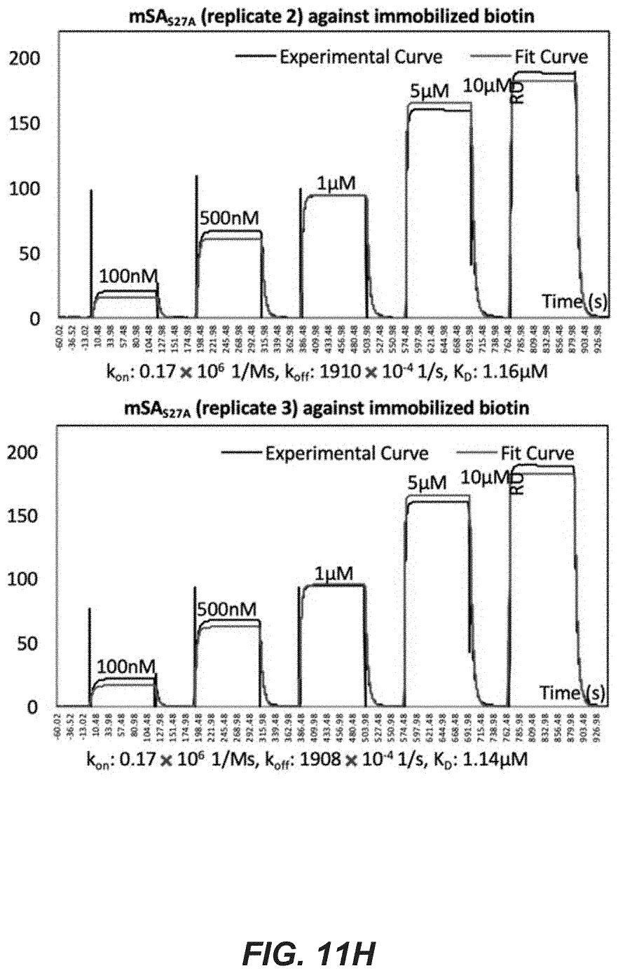

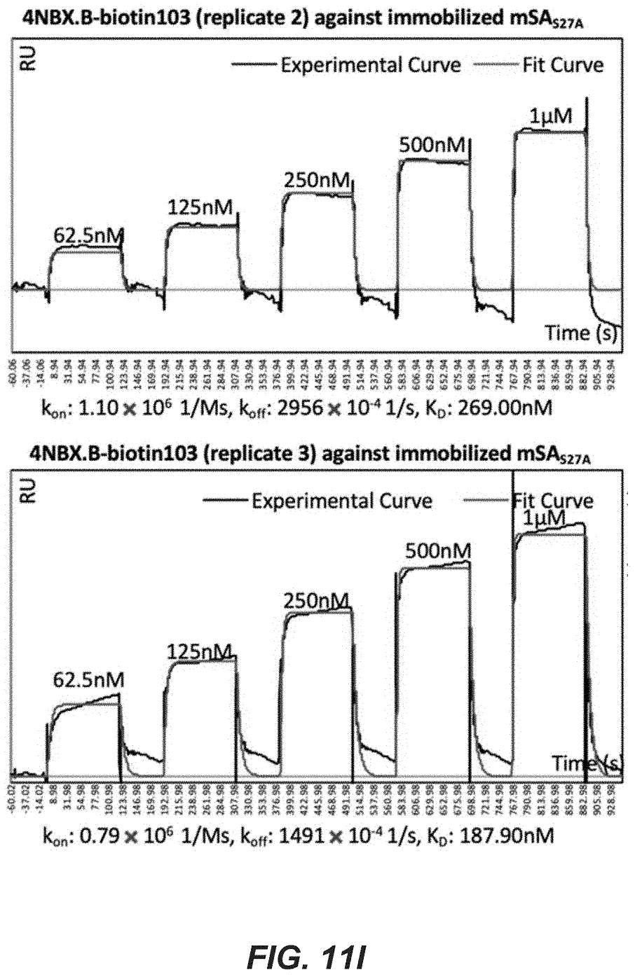

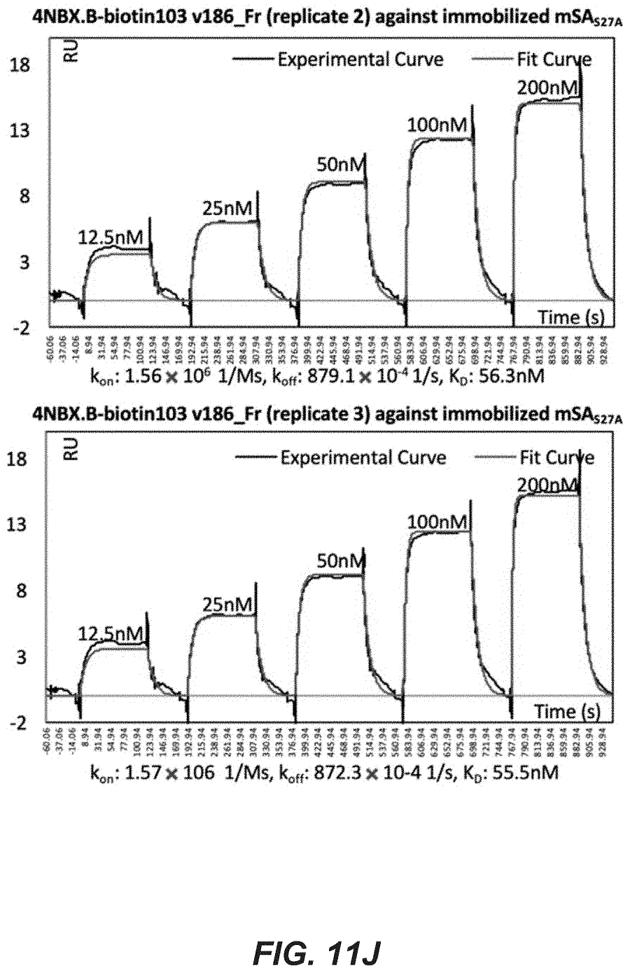

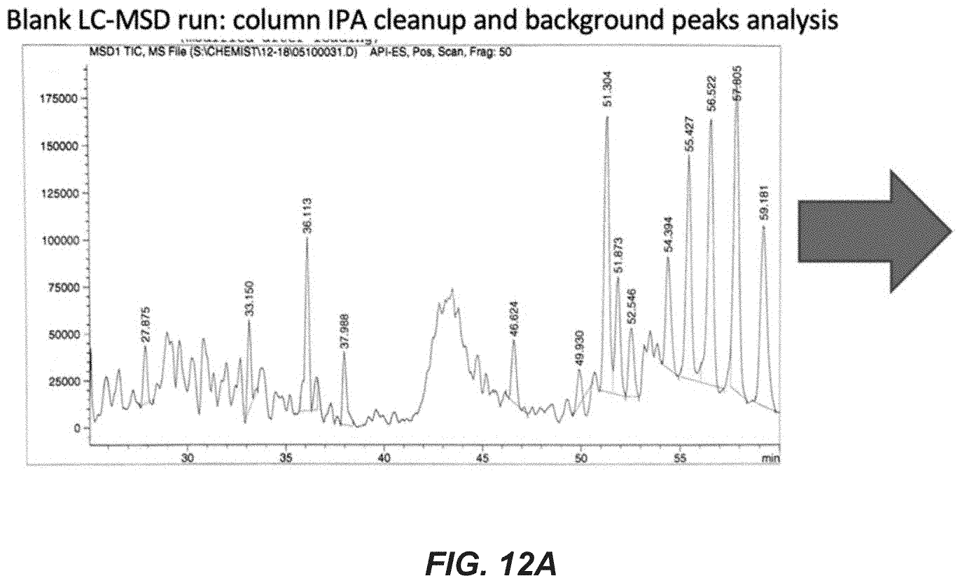

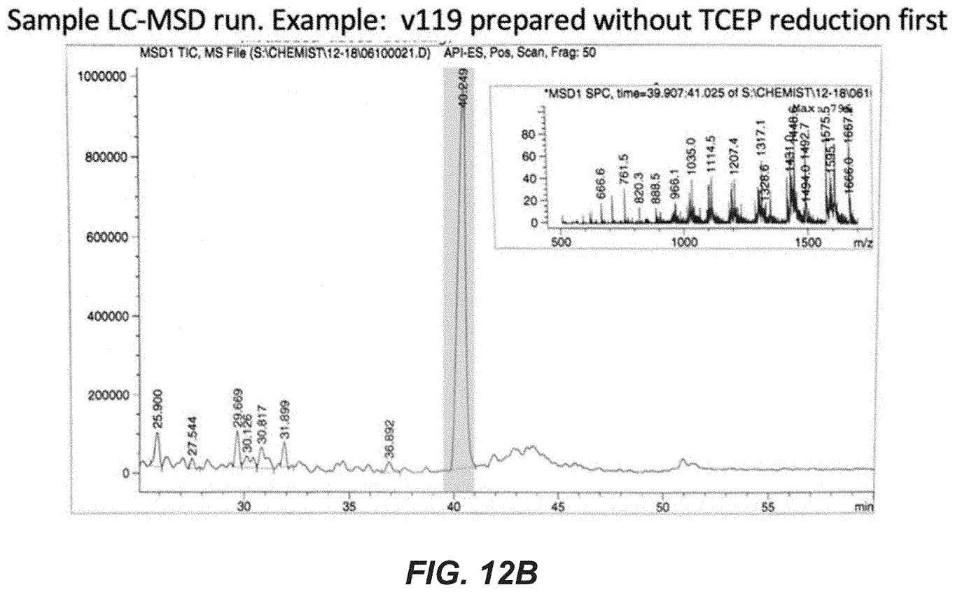

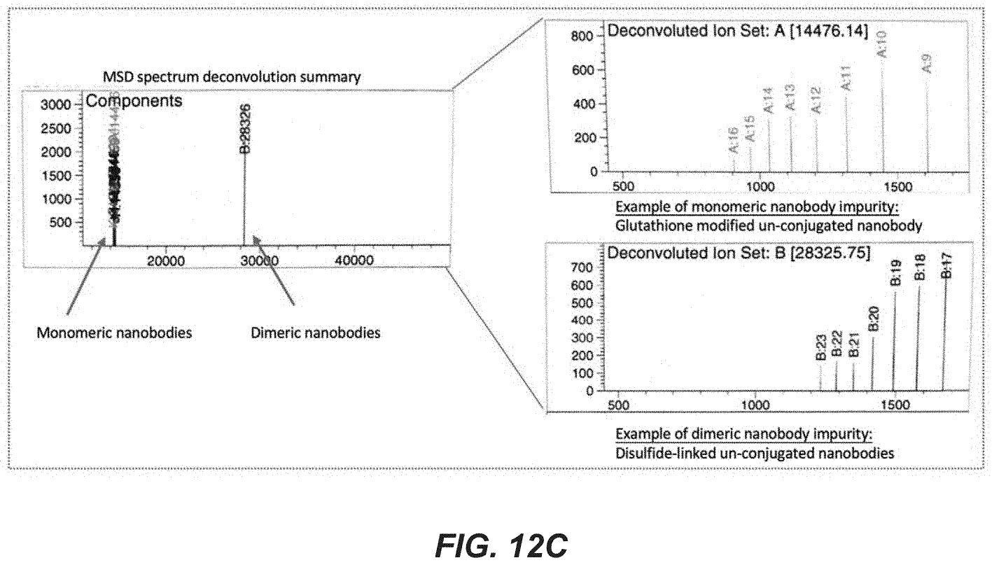

To further investigate the functionally-relevant structural features of v186 and v186_Fr, additional triplicate 100 ns MD simulations of v186 and v186_Fr against mSA_{WT} were performed. In general, during the simulations both the overall binding geometry of the conjugates and the conformation of the biotin103 side chain remained constant with small structural RMSDs (FIG. 10A-FIG. 10B, FIG. 4B). The 4NBX.B nanobody scaffold has two solvent-inaccessible clusters of hydrophobic residues in the framework, one being the β-barrel core and another shielded by the CDR3 loop (FIG. 4A). Stable solvent inaccessibility and packing of hydrophobic patches is usually correlated with protein folding stability, which is in turn related to aggregation. For the majority of time in the MD simulations, the solvent-accessible area for the two hydrophobic clusters of both v186 and v186_Fr was distributed around similarly-low values, indicating that both variants should be generally foldable (FIG. 4A). However, in contrast to v186_Fr, v186 displayed apparent sub-populations whose hydrophobic core and CDR3-shielded hydrophobic residues were significantly more solvent-accessible, indicating possible structural instability that agrees with the expected stabilization effects of F37Y and A12V in v186_Fr (FIG. 4A). Additional analysis of the v186_Fr/mSA_{WT} interface from the simulations indicates high shape complementarity, large buried interface area, and close interface distance that remained generally constant along the timescale, in agreement with the measured sub-nanomolar affinity (FIG. 4B-FIG. 4E). Overall, the design calculation, experimental data, and MD simulations are well-correlated with each other.

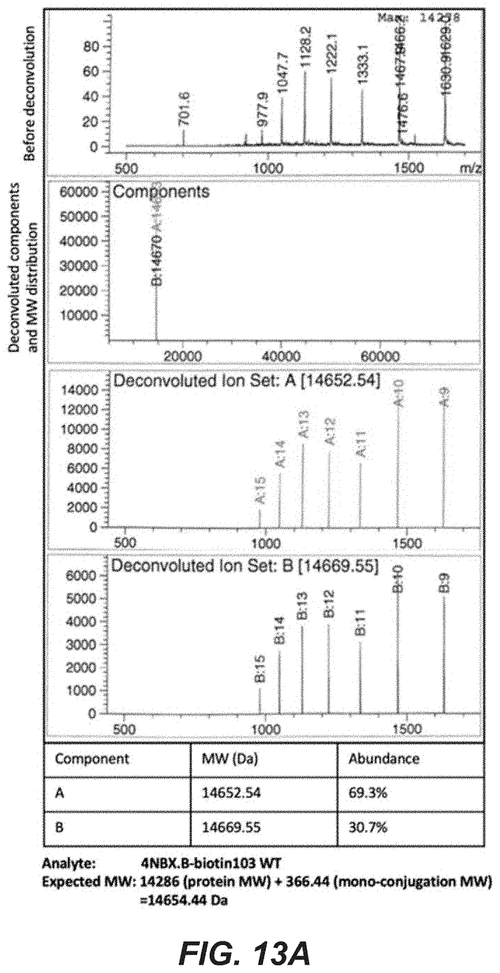

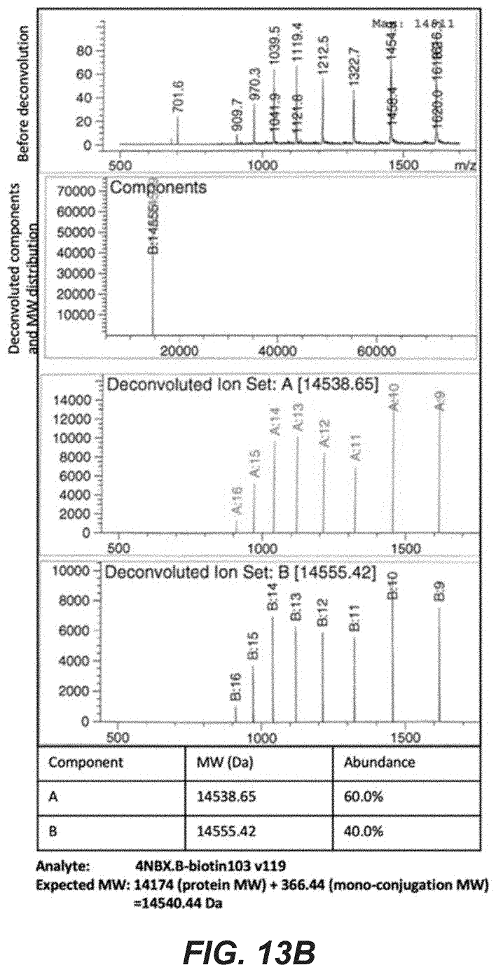

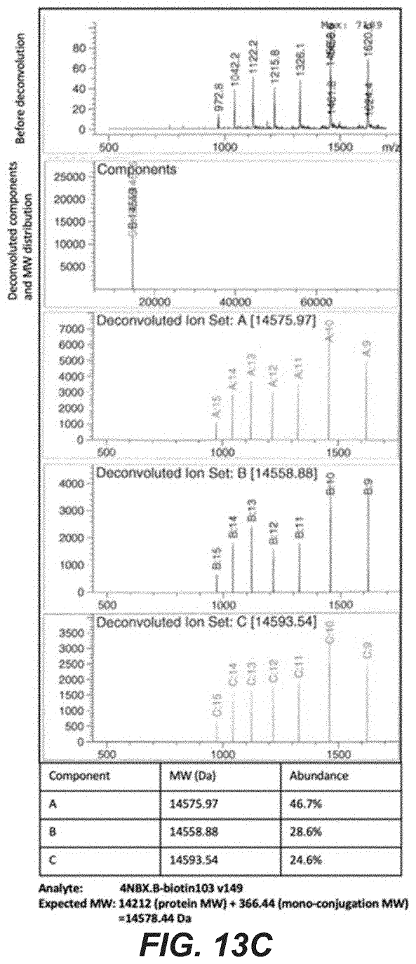

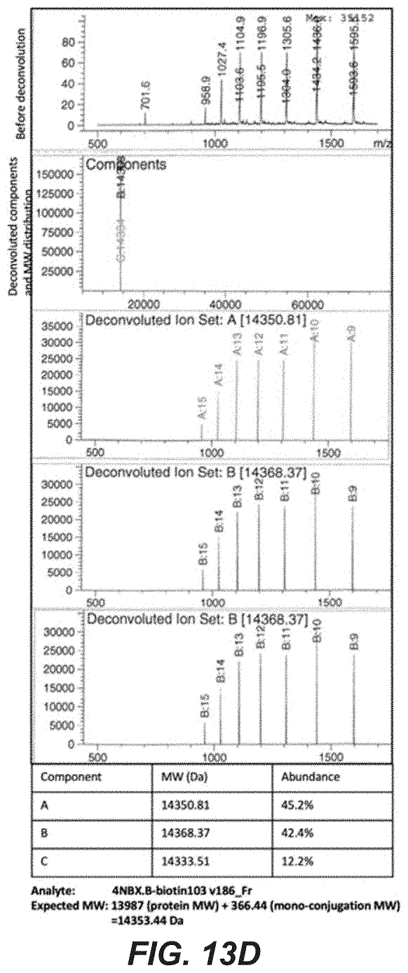

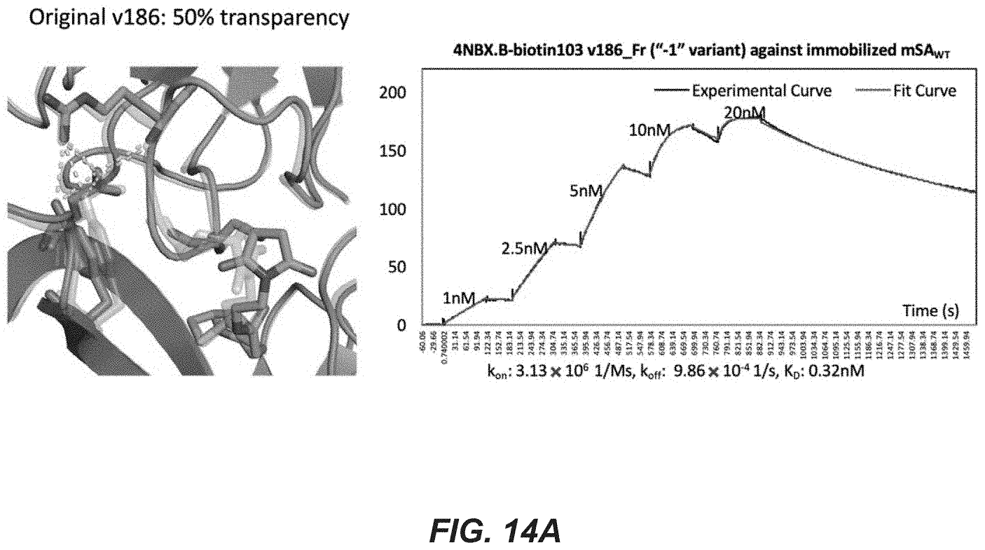

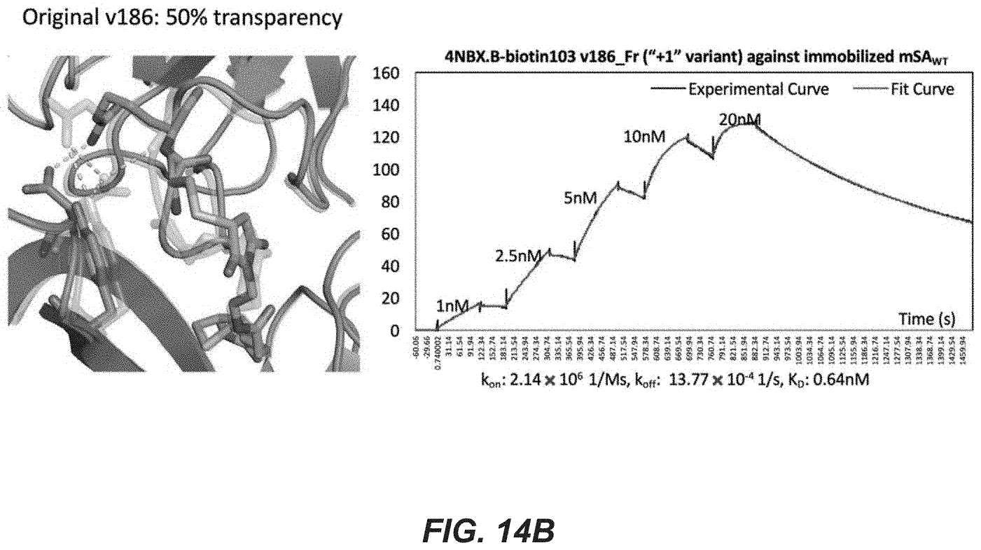

Affinity and kinetics estimation of 4NBX.B-biotin103 WT, v119, v149, and v186_Fr were performed in biological triplicates. To make sure the prepared conjugates homogeneously harbor one biotin-maleimide “sidechain” per nanobody molecule, intact-protein mass spectrometry (MS) was used to analyze one of the SPR-measured triplicates for each of the above-mentioned nanobody-biotin variants, as it was reasoned that one replicate should be representative given the small batch-to-batch variations in measured affinities (FIG. 5A-FIG. 5B). Deconvolution of MS spectra only returned components with molecular weights (MWs) within 20 Da from the expected values of mono-biotin conjugates, while each conjugated biotin-maleimide “sidechain” would add an additional mass of 366 Da, indicating that all tested materials are mono-conjugated with biotin C2 maleimide (FIG. 13A-FIG. 13D). Subpopulations with ˜+/−17 Da from the expected MWs were observed, and could be contributed by deamidation of Gln/Asn groups, ring-open products of succinimides, or ion adducts (FIG. 13A-FIG. 13D). To test how the binding of v186_Fr to mSA_{WT} would respond to small geometry perturbation, two variants were created where biotin103 was moved respectively to +1 (SEQ ID NO:7, Table 4) and −1 (SEQ ID NO:8, Table 4) positions on CDR3 (FIG. 14A-FIG. 14B). Modeling of the biotin103 sidechain in the +1 and −1 variants suggests that the mutations would exert tolerable influence on the originally designed v186_Fr/mSA_{WT} interactions (FIG. 14A-FIG. 14B). Measured affinities of the +1 and −1 variants against mSA_{WT} were minimally different from the wildtype v186_Fr, in agreement to the modeling (FIG. 14A-FIG. 14B). Interestingly, the +1 variant is apparently a 2-fold weaker binder than the −1 variant, while the modeling also suggests that the +1 mutation would be more detrimental than the −1 as its presence would likely cause the affinity-contributing R105 sidechain to be more difficult to adopt the designed conformation (FIG. 14A-FIG. 14B).

### Summary and Further Testing of a Computational Workflow for Creating Synergistically-Binding Nanobody Small-Molecule Conjugates

A non-limiting exemplary design process described herein includes: docking a library of nanobody structures with diverse CDR sequences and conformations onto a desired target in complex with the to-be-conjugated small molecule, filtering binding poses to preserve ones that closely resemble the original binding mode of the original nanobody scaffold, screening the rotamer library of the conjugated small molecule onto the poses to identify most tolerable conjugation plan, and finally re-designing the sequences of both the nanobody CDR loops and framework to improve binding affinity, kinetics, and overall stability (FIG. 5D).

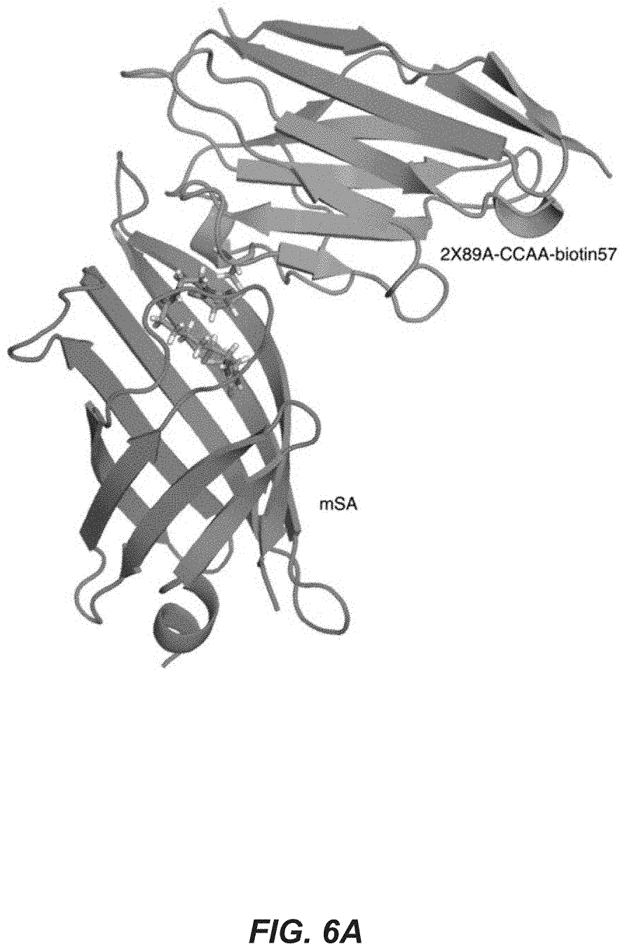

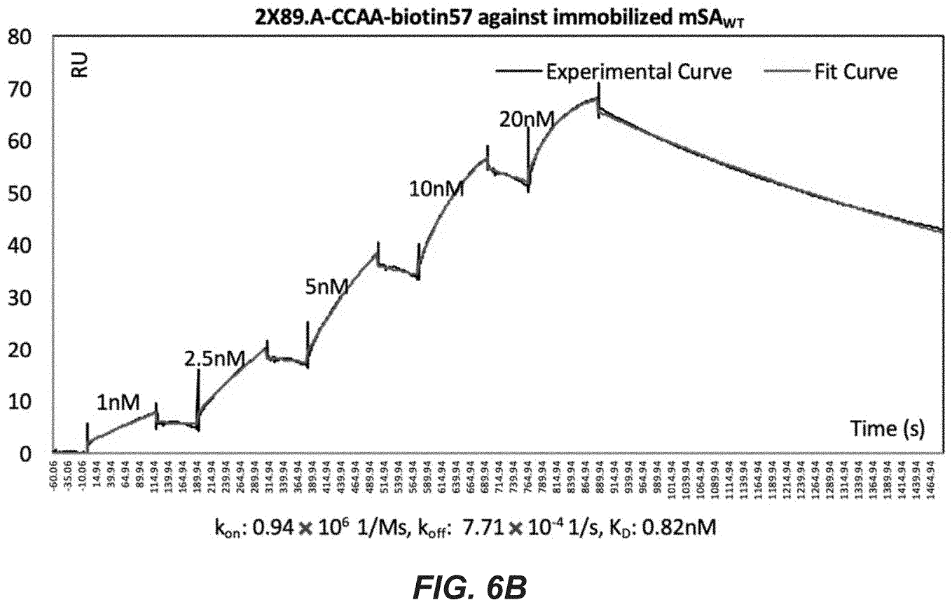

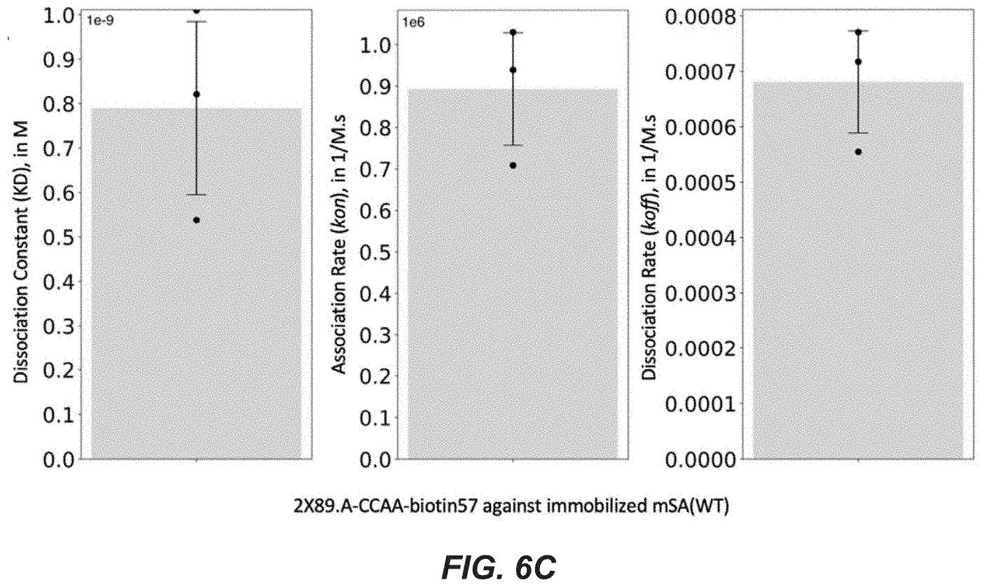

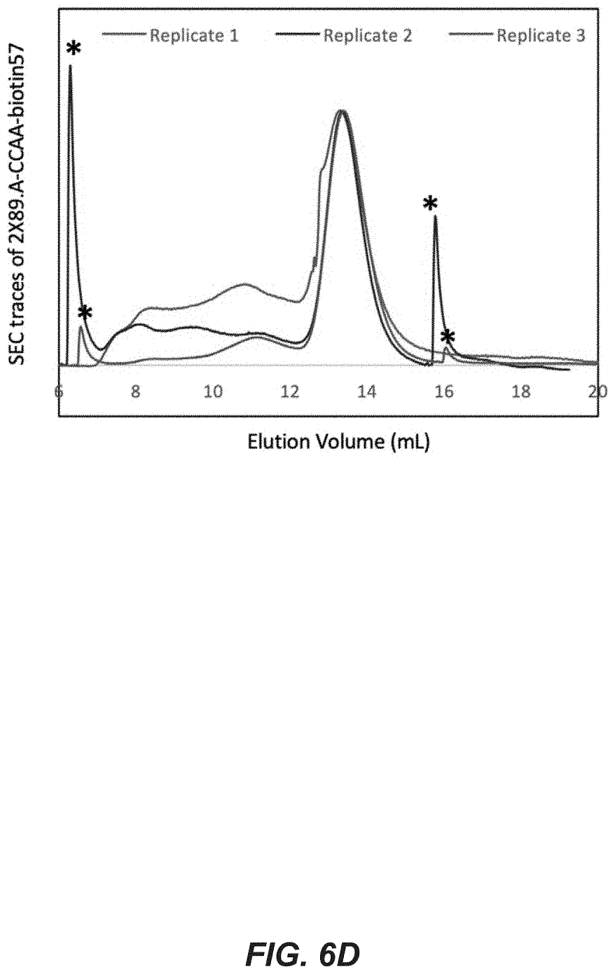

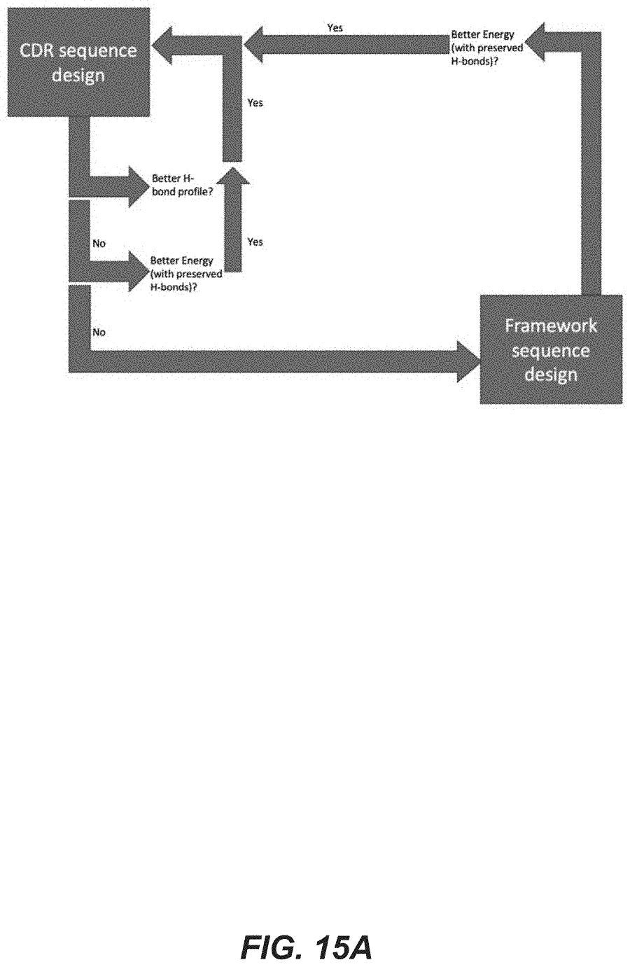

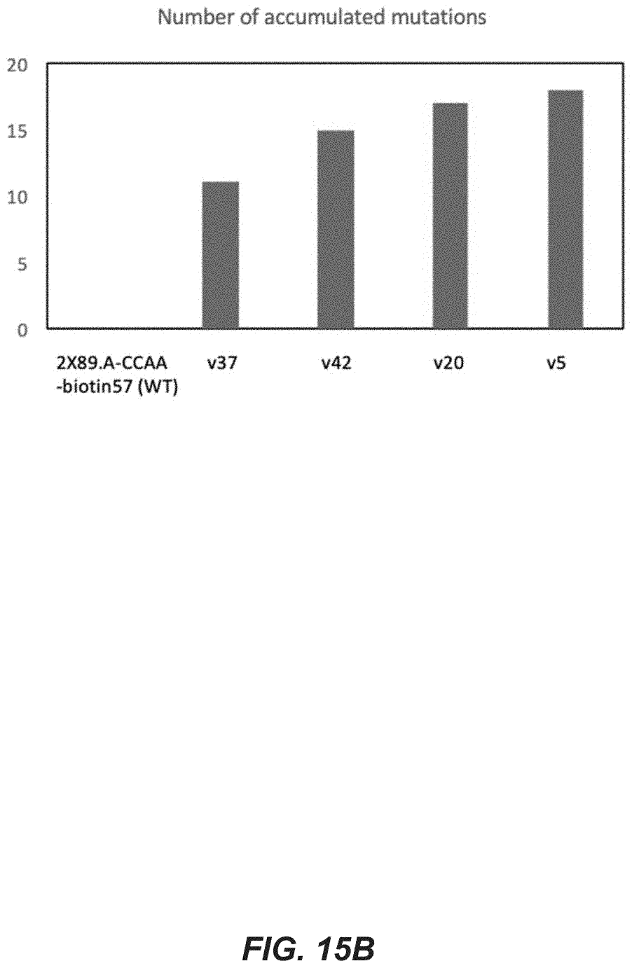

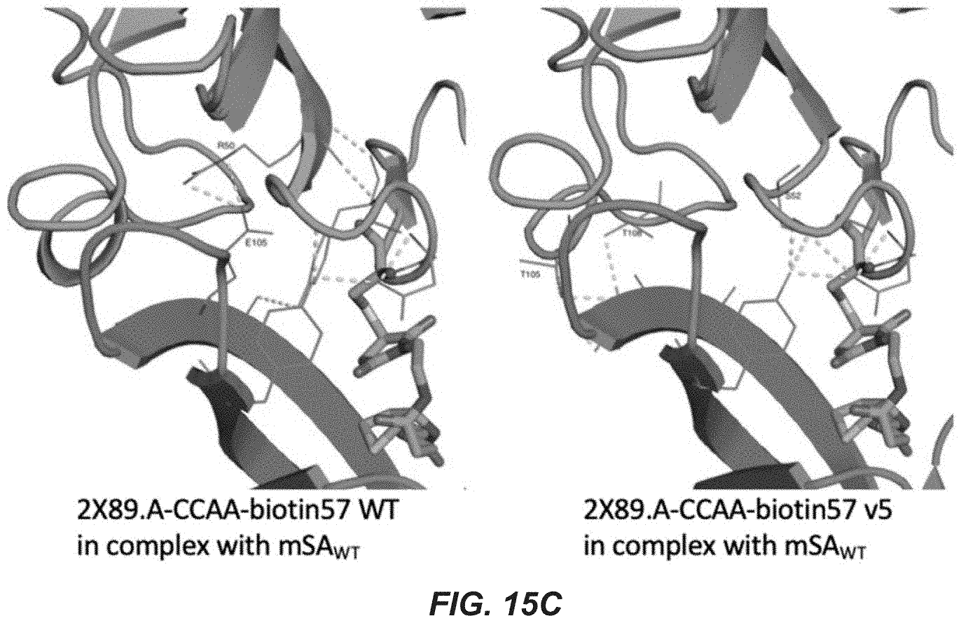

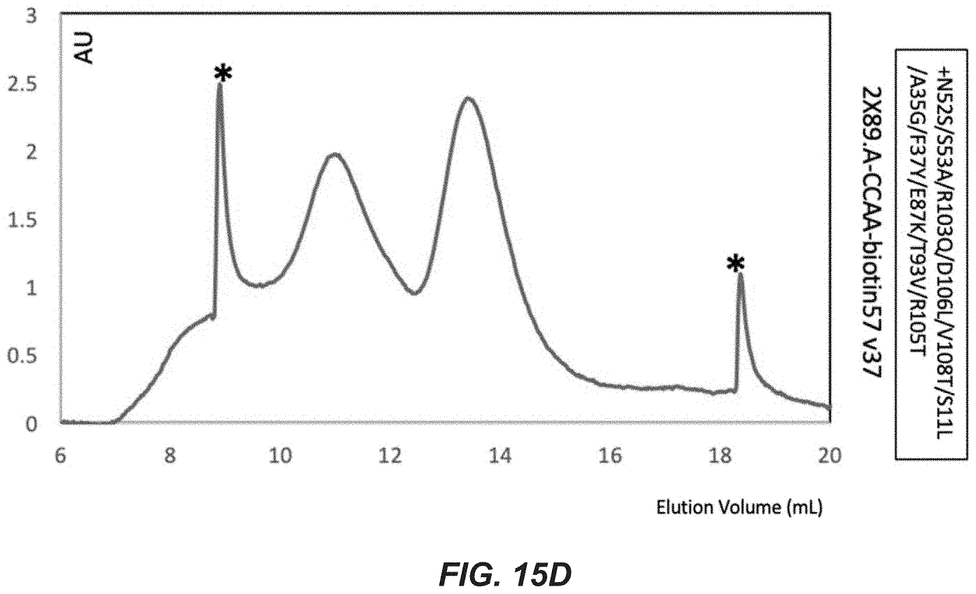

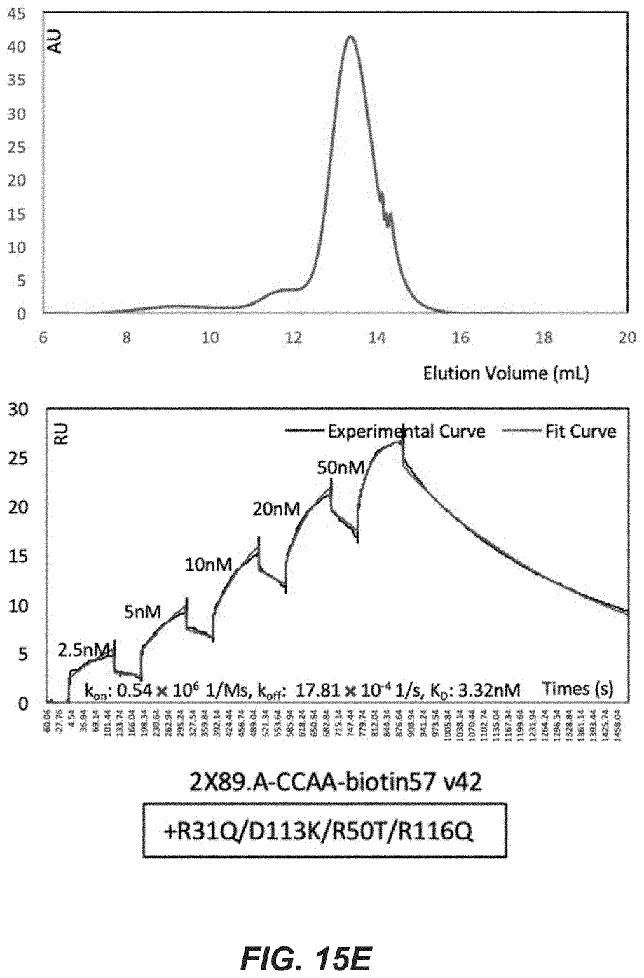

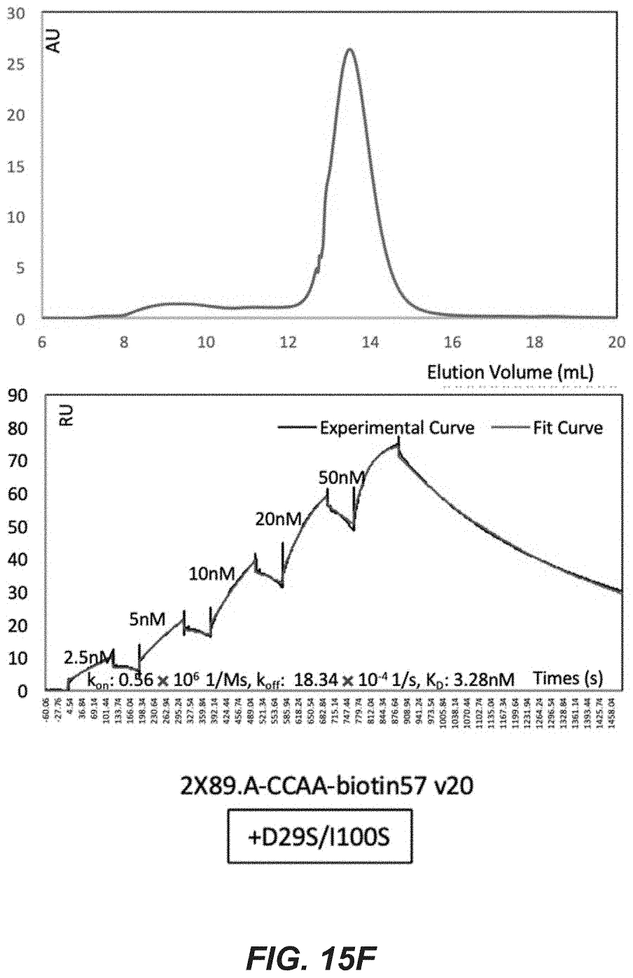

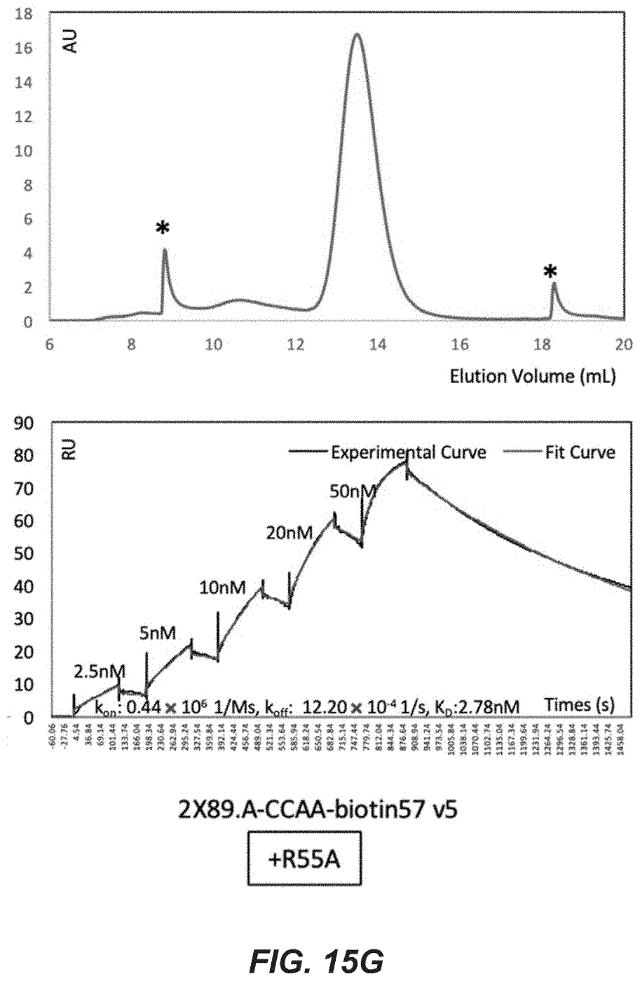

Because 4NBX.B was not obtained by directly docking nanobody scaffolds against mSA, the docking, filtering, and rotamer screening steps were re-performed on mSA, and selected a different scaffold, 2X89.A (SEQ ID NO:9, Table 4), with biotin conjugated to site 57 (FIG. 6A). Similar to 4NBX.B-biotin103 v186_Fr, the selected pose of 2X89.A was predicted to interact with mSA_{WT} through a R-E interaction, together with other potential intermolecular H-bonds (FIG. 15C left panel). Since the original 2X89.A has an additional intra-CDR disulfide bond, to avoid over conjugation, the disulfide bond was replaced by two alanine residues. The resulted final conjugate, 2X89.A-CCAA-biotin57 (SEQ ID NO:10, Table 4) binds to mSA_{WT} with a K_{D} of 0.8±0.2 nM, and remarkably, a k_{off} that is slightly better than the best designed 4NBX.B variant v186_Fr (FIG. 6B-FIG. 6C). 2X89.A-CCAA-biotin57 aggregated obviously on SEC (FIG. 6D). To reduce aggregation, a rudimentary sequence design pipeline was constructed that sways between CDR and framework design based on our previous experience on designing 4NBX.B conjugates, and applied the pipeline on 2X89.A (FIG. 15A). Top-ranked variants along the six rounds of CDR designs and one round of framework design showed first worsened then improved aggregation profile, which was eventually better than the 2X89.A-CCAA-biotin57 parent conjugate in variant v5 (SEQ ID NO:14, Table 4) that accumulated 18 mutations (FIG. 15B-FIG. 15G). Some predicted H-bonds in the parent conjugate were disrupted along the design process while additional H-bonds were predicted to form in other places (FIG. 15C). Although the total number of predicted intermolecular H-bonds stayed roughly identical along the design process, the binding strengths of sequence-designed variants were lower than the parent as the predicted H-bonds shuffle to different locations, suggesting that a more accurate comparison of different H-bond profiles would be beneficial to improve design accuracy (FIG. 15C-15G). However, the slow dissociation rate in the parent conjugate was relatively preserved in the final variant v5 (FIG. 15D-FIG. 15G).

TABLE 3
TOP 20 SEQUENCE OUTPUTS FROM FRAMEWORK DESIGN
ON 4NBX.B-BIOTIN103 V149. (RANKED BY ENERGY SCORE)

| | Energy | | Mutations: chain name and |
|---|---|---|---|
| Ranking | Score | Note | accepted residue |
| 1 | −482.29049 | | WT |
| 2 | −481.67807 | | B_37Y |
| 3 | −481.6627 | | B_5Q + B_12V |
| 4 | −481.65469 | A12V/F37Y | B_12V + B_37Y |
| | | mutations | |
| 5 | −481.51508 | | B_12V + B_35G |
| 6 | −481.5122 | | B_5Q |
| 7 | −481.48205 | | B_35G |
| 8 | −481.38994 | | B_35G + B_37Y |
| 9 | −481.32699 | | B_5Q + B_37Y |
| 10 | −481.13866 | | B_40A |
| 11 | −481.12555 | | B_12V |
| 12 | −480.83661 | | B_35G + B_40A |
| 13 | −480.82825 | | B_12V + B_37Y + B_40A |
| 14 | −480.50684 | | B_5Q + B_35G + B_37Y |
| 15 | −480.44015 | | B_5Q + B_12V + B_40A |
| 16 | −480.42843 | | B_12V + B_35G + B_37Y |
| 17 | −480.3031 | | B_5Q + B_12V + B_37Y + B_40A |
| 18 | −480.19273 | | B_5Q + B_12V + B_35G + B_40A |
| 19 | −480.16999 | | B_5Q + B_35G + B_40A |
| 20 | −480.16866 | | B_12V + B_35G + B_37Y + |
| | | | B_40A |

TABLE 4
AMINO ACID SEQUENCES OF PROTEINS

| | | Mutations | SEQ |
|---|---|---|---|
| | | (relative | ID |
| Name | Sequence | to WT) | NO |
| 4NBX.B- | QVQLQESGGGLAQ | N/A | 1 |
| WT | AGGSLRLSCAASG | | |
| | RTFSMDPMAWFRQ | | |
| | PPGKEREFVAAGS | | |
| | STGRTTYYADSVK | | |
| | GRFTISRDNAKNT | | |
| | VYLQMNSLKPEDT | | |
| | AVYYCAAAPYGAN | | |
| | WYRDEYAYWGQGT | | |
| | QVTVSSHHHHHH | | |
| 4NBX.B_ | QVQLQESGGGLAQ | A103C | 2 |
| A103C | AGGSLRLSCAASG | | |
| | RTFSMDPMAWFRQ | | |
| | PPGKEREFVAAGS | | |
| | STGRTTYYADSVK | | |
| | GRFTISRDNAKNT | | |
| | VYLQMNSLKPEDT | | |
| | AVYYCAAAPYGCN | | |
| | WYRDEYAYWGQGT | | |
| | QVTVSSHHHHHH | | |
| 4NBX.B- | QVQLQESGGGLAQ | A103C/ | 3 |
| biotin103 | AGGSLRLSCAASG | M31H/ | |
| v119 | RTFSHAPMAWFRQ | D32A/ | |
| | PPGKEREFVAAGS | N104S/ | |
| | STGRTTYYADSVK | W105 | |
| | GRFTISRDNAKNT | H | |
| | VYLQMNSLKPEDT | | |
| | AVYYCAAAPYGCS | | |
| | HYRDEYAYWGQGT | | |
| | QVTVSSHHHHHH | | |
| 4NBX.B- | QVQLQESGGGLAQ | A103C/ | 4 |
| biotin103 | AGGSLRLSCAASG | M31R/ | |
| v149 | RTFSRSPMAWFRQ | D32S/ | |
| | PPGKEREFVAAGS | N104A/ | |
| | STGRTTYYADSVK | W105 | |
| | GRFTISRDNAKNT | R | |
| | VYLQMNSLKPEDT | | |
| | AVYYCAAAPYGCA | | |
| | RYRDEYAYWGQGT | | |
| | QVTVSSHHHHHH | | |
| 4NBX.B- | QVQLQESGGGLAQ | A103C/ | 5 |
| biotin103 | AGGSLRLSCAASG | M31R/ | |
| v186 | RTFSRSPMAWFRQ | D32S/ | |
| | PPGKEREFVAAGS | N104A/ | |
| | STGTTTYYADSVK | W105R/ | |
| | GRFTISRDNAKNT | Y101L/ | |
| | VYLQMNSLKPEDT | R107F/ | |
| | AVYYCAAAPLGCA | R56T/ | |
| | RKFAESAYWGQGT | Y106K/ | |
| | QVTVSSHHHHHH | D108A/ | |
| | | Y110S | |
| 4NBX.B- | QVQLQESGGGLVQ | A103C/ | 6 |
| biotin103 | AGGSLRLSCAASG | M31R/ | |
| v186_Fr | RTFSRSPMAWYRQ | D32S/ | |
| | PPGKEREFVAAGS | N104A/ | |
| | STGTTTYYADSVK | W105R/ | |
| | GRFTISRDNAKNT | Y101L/ | |
| | VYLQMNSLKPEDT | R107F/ | |
| | AVYYCAAAPLGCA | R56T/ | |
| | RKFAESAYWGQGT | Y106K/ | |
| | QVTVSSHHHHHH | D108A/ | |
| | | Y110S/ | |
| | | A12V/ | |
| | | F37Y | |
| 4NBX.B- | QVQLQESGGGLVQ | M31R/ | 7 |
| biotin103 | AGGSLRLSCAASG | D32S/ | |
| v186_ | RTFSRSPMAWYRQ | N104C/ | |
| Fr + 1 | PPGKEREFVAAGS | W105R/ | |
| | STGTTTYYADSVK | YI01L/ | |
| | RGFTISRDNAKNT | R107F/ | |
| | VLYQMNSLKPEDT | R56T/ | |
| | AVYYCAAAPLGAC | Y106K/ | |
| | FRKAESAYWGQGT | D108A/ | |
| | QVTVSSHHHHHH | Y110S/ | |
| | | A12V/ | |
| | | F37Y | |
| 4NBX.B- | QVQLQESGGGLVQ | G102C/ | 8 |
| biotin103 | AGGSLRLSCAASG | M31R/ | |
| v186_ | RTFSRSPMAWYRQ | D32S/ | |
| Fr − 1 | PPGKEREFVAAGS | N104A/ | |
| | STGTTTYYADSVK | W105R/ | |
| | GRFTISRDNAKNT | Y101L/ | |
| | VYLQMNSLKPEDT | R107F/ | |
| | AVYYCAAAPLCAA | R56T/ | |
| | RKFAESAYWGQGT | Y106K/ | |
| | QVTVSSHHHHHH | D108A/ | |
| | | Y110S/ | |
| | | A12V/ | |
| | | F37Y | |
| 2X89.A- | QVQLQESGGGSVQ | N/A | 9 |
| WT | AGGSLRLSCAASG | | |
| | YTDSRYCMAWFRQ | | |
| | APGKEREWVARIN | | |
| | SGRDITYYADSVK | | |
| | GRFTFSQDNAKNT | | |
| | VYLQMDSLEPEDT | | |
| | ATYYCATDIPLRC | | |
| | RDIVAKGGDGFRY | | |
| | WGQGTQVTVSSHH | | |
| | HHHH | | |
| 2X89.A_ | QVQLQESGGGSVQ | I57C/ | 10 |
| CCAA_I | AGGSLRLSCAASG | C33A/ | |
| 57C | YTDSRYAMAWFRQ | C104A | |
| | APGKEREWVARI | | |
| | NSGRDCTYYADSV | | |
| | KGRFTFSQDNAKN | | |
| | TVYLQMDSLEPED | | |
| | TATYYCATDIPLR | | |
| | ARDIVAKGGDGFR | | |
| | YWGQGTQVTVSSH | | |
| | HHHHH | | |
| 2X89.A- | QVQLQESGGGLVQ | I57C/ | 11 |
| CCAA- | AGGSLRLSCAASG | C33A/ | |
| biotin57 | YTDSRYAMGWYRQ | C104A/ | |
| v37 | APGKEREWVARIS | N52S/ | |
| | AGRDCTYYADSVK | S53A/ | |
| | GRFTFSQDNAKNT | R103Q/ | |
| | VYLQMDSLKPEDT | D106L/ | |
| | AVYYCATDIPLQA | V108T/ | |
| | TLITAKGGDGFRY | S11L/ | |
| | WGQGTQVTVSSHH | A35G/ | |
| | HHHH | F37Y/ | |
| | | E87K/ | |
| | | T93V/ | |
| | | R105T | |
| 2X89.A- | QVQLQESGGGLVQ | I57C/ | 12 |
| CCAA- | AGGSLRLSCAASG | C33A/ | |
| biotin57 | YTDSQYAMGWYRQ | C104A/ | |
| v42 | APGKEREWVATIS | N52S/ | |
| | AGRDCTYYADSVK | S53A/ | |
| | GRFTFSQDNAKNT | R103Q/ | |
| | VYLQMDSLKPEDT | D106L/ | |
| | AVYYCATDIPLQA | V108T/ | |
| | TLITAKGGKGFQY | S11L/ | |
| | WGQGTQVTVSSHH | A35G/ | |
| | HHHH | F37Y/ | |
| | | E87K/ | |
| | | T93V/ | |
| | | R105T/ | |
| | | R31Q/ | |
| | | D113K/ | |
| | | R50T/ | |
| | | R116Q | |
| 2X89.A- | QVQLQESGGGLVQ | 157C/ | 13 |
| CCAA- | AGGSLRLSCAASG | C33A/ | |
| biotin57 | YTSSQYAMGWYRQ | C104A/ | |
| v20 | APGKEREWVATIS | N52S/ | |
| | AGRDCTYYADSVK | S53A/ | |
| | GRFTFSQDNAKNT | R1 | |
| | VYLQMDSLKPEDT | 03Q/ | |
| | AVYYCATDSPLQA | D106L/ | |
| | TLITAKGGKGFQY | V108T/ | |
| | WGQGTQVTVSSHH | S11L/ | |
| | HHHH | A35G/ | |
| | | F37Y/ | |
| | | E87K/ | |
| | | T93V/ | |
| | | R105/ | |
| | | VR31Q/ | |
| | | D113K/ | |
| | | R50T/ | |
| | | R116Q/ | |
| | | D29S/ | |
| | | I100S | |
| 2X89.A- | QVQLQESGGGLVQ | I57C/ | 14 |
| CCAA- | AGGSLRLSCAASG | C33A/ | |
| biotin57 | YTSSQYAMGWYRQ | C104A/ | |
| v5 | APGKEREWVATIS | N52S/ | |
| | AGADCTYYADSVK | S53A/ | |
| | GRFTFSQDNAKNT | R103Q/ | |
| | VYLQMDSLKPEDT | D106L/ | |
| | AVYYCATDSPLQA | V108T/ | |
| | TLITAKGGKGFQY | S11L/ | |
| | WGQGTQVTVSSHH | A35G/ | |
| | HHHH | F37Y/ | |
| | | E87K/ | |
| | | T93V/ | |
| | | R105T/ | |
| | | R31Q/ | |
| | | D113K/ | |
| | | R50T/ | |
| | | R116Q/ | |
| | | D29S/ | |
| | | 1100S/ | |
| | | R55A | |
| SA(core | GITGTWYNQLGST | N/A | 15 |
| 16-133)- | FIVTAGADGALTG | | |
| WT | TYESAVGNAESRY | | |
| | VLTGRYDSAPATD | | |
| | GSGTALGWTVAWK | | |
| | NNYRNAHSATTWS | | |
| | GQYVGGAEARINT | | |
| | QWLLTSGTTEANA | | |
| | WKSTLVGHDTFTK | | |
| | V | | |
| SA(core | GITGTWYNQLGST | S45A/ | 16 |
| 16-133) | FIVTAGADGALTG | T90A/ | |
| S45A/ | TYEAAVGNAESRY | D128A | |
| T90A/ | YLTGRYDSAPATD | | |
| D128A | GSGTALGWTVAWK | | |
| | NNYRNAHSAATWS | | |
| | GQYVGGAEARINT | | |
| | QWLLTSGTTEANA | | |
| | WKSTLVGHATFTK | | |
| | V | | |
| mSA_WT | HHHHHHSQDLASA | N/A | 17 |
| | EAGITGTWYNQSG | | |
| | STFTVTAGADGNL | | |
| | TGQYENRAQGTGC | | |
| | QNSPYTLTGRYNG | | |
| | TKLEWRVEWNNST | | |
| | ENCHSRTEWRGQY | | |
| | QGGAEARINTQWN | | |
| | LTYEGGSGPATEQ | | |
| | GQDTFTKVKPSAA | | |
| | SGSDYKDDDDK | | |
| mSA_{S27A} | HHHHHHSQDLASA | S27A | 18 |
| | EAGITGTWYNQSG | | |
| | ATFTVTAGADGNL | | |
| | TGQYENRAQGTGC | | |
| | QNSPYTLTGRYNG | | |
| | TKLEWRVEWNNST | | |
| | ENCHSRTEWRGQY | | |
| | QGGAEARINTQWN | | |
| | LTYEGGSGPATEQ | | |
| | GQDTFTKVKPSAA | | |
| | SGSDYKDDDDK | | |

Using mSA/biotin system, the present example demonstrated that a complementary immunoglobulin domain conjugating to the small molecule can be designed entirely by computational methods to bind tighter against the target, for example with only structural information of a small molecule binding to its target. The method disclosed herein can bridge the two worlds of small molecules and biologics. The binding interface for the designed conjugates comprise of both an ultra-deep pocket that is uncommon for antibodies, and broad contacting interface that is uncommon for small molecules. Therefore, the chemical space and target space of traditional molecular recognition agents can be expanded in this manner, offering new potential solutions to a wide range of challenges, such as reutilizing failed small molecules or tackling undruggable targets in pharmaceutical development. The results disclosed herein showed that the affinity, kinetics, and stability of the conjugates can be designed in a step-wise manner, indicating that the development process is highly tunable and multiple physicochemical properties can be simultaneously optimized.

The design method disclosed herein can be used for therapeutically-relevant targets. In some embodiments, the workflow can be used with virtually-docked small-molecule/target complexes. In some embodiments, specificity, affinity or both can be altered. In some embodiments, virtually recombining structural fragments can help affinity maturation of computationally designed antibodies. In some embodiments, specifically-tailored algorithms that put more bias in the formation of hydrogen-bonding networks can be useful to the affinity and specificity of designed protein/protein interfaces. In some embodiments, additional loop-modeling methods and ensemble design can be used to adjust binding poses for the conjugates, and engineer specificities.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for designing antibody-small-molecule conjugates, comprising:
(a) receiving three-dimensional coordinates for a crystal structure of a target protein in complex with a small molecule;
(b) docking a plurality of antibody structures onto the crystal structure, wherein each of the plurality of antibody structures has a different complementarity-determining region (CDR) conformations from each other and thereby different binding pose against the target protein surface;
(c) identifying one or more of the plurality of antibody structures with binding poses that accommodate both the CDR binding poses and the target protein-small molecule interaction;
(d) screening a rotamer library of the conjugated small molecule onto the CDR binding poses identified in (c) to identify a conjugation plan comprising a selected conjugation site on the antibody to which the small molecule is conjugated to; and
(e) adjusting the sequences of the antibody CDR loops, the antibody framework or both in the conjugation plan identified in (d) to generate an antibody capable of forming an antibody-small-molecule conjugate with the small molecule, wherein the antibody-small-molecule conjugate has a higher binding affinity to the target protein as compared to the small molecule alone or to a reference antibody-small-molecule conjugate.

2. The method of claim 1, wherein identifying the antibody structures in step (c) comprises performing loop-modeling on docketed poses.

3. The method of claim 2, wherein performing loop-modeling on docketed poses comprises searching naturally occurring antibody CDR binding conformation.

4. The method of claim 1, wherein the binding affinity of the antibody-small-molecule conjugate to the target protein is at least two fold higher than the binding affinity of the small molecule alone or the reference antibody-small-molecule conjugate.

5. The method of claim 1, wherein the binding affinity of the antibody-small-molecule conjugate to the target protein is at least five fold higher than the binding affinity of the small molecule alone or the reference antibody-small-molecule conjugate.

6. The method of claim 1, wherein the target protein-small molecule interaction in (c) comprises an interaction between the small molecule and a variant of the target protein.

7. The method of claim 1, wherein the rotamer library of the conjugated small molecule is a rotamer library of cysteine-conjugated side chain.

8. The method of claim 1, wherein adjusting the sequence of the antibody CDR loops in (e) comprises adjusting the sequence of the antibody CDR residues close to the binding sites of the small molecule to the protein target.

9. The method of claim 1, wherein the antibody is a nanobody, a monoclonal antibody, and a combination thereof.

10. The method of claim 1, wherein the antibody-small-molecule conjugate has improved kinetics, metabolic stability, circulation half-life, solubility, systemic toxicity, or any combination of, compared to the small molecule alone.

11. The method of claim 1, wherein the antibody-small-molecule conjugate has improved binding specificity to the protein target compared to the small molecule alone.

12. The method of claim 1, further comprising adjusting the sequences of the antibody CDR loops to increase H-bond formation between the antibody and the target protein surface.

13. The method of claim 1, wherein the binding surface for the designed antibody-small-molecule conjugate to the target protein comprises an ultra-deep pocket, broad contacting interface, or both.

14. The method of claim 1, wherein the small molecule is a therapeutic agent.

15. The method of claim 14, wherein the therapeutic agent is a cancer drug or a cytotoxic drug.

16. The method of claim 1, wherein the target protein is a tumor antigen,

17. The method of claim 1, wherein the antibody-small-molecule conjugate is an antibody-drug conjugate (ADC).

18. The method of claim 17, wherein the ADC is Gemtuzumab ozogamicin, Brentuximab vedotin, Trastuzumab emtansine, Inotuzumab ozogamicin, Polatuzumab vedotin, Enfortumab vedotin, Trastuzumab deruxtecan, Sacituzumab govitecan, Belantamab mafodotin, Moxetumomab pasudotox, or Loncastuximab tesirine.

19. The method of claim 1, wherein two or more of the plurality of antibody structures have different CDR sequences.

20. The method of claim 1, further comprising producing the designed antibody-small-molecule conjugate.


SEQUENCE LISTING (18 sequences)

SEQ ID NO: 1 - protein, 129 aa, Artificial Sequence
  4NBX.B-WT
QVQLQESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWYRDEYAYWGQGTQVTVSSHHHHHH

SEQ ID NO: 2 - protein, 129 aa, Artificial Sequence
  4NBX.B_A103C
QVQLQESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGCNWYRDEYAYWGQGTQVTVSSHHHHHH

SEQ ID NO: 3 - protein, 129 aa, Artificial Sequence
  4NBX.B-biotin103 v119
QVQLQESGGGLAQAGGSLRLSCAASGRTFSHAPMAWFRQPPGKEREFVAAGSSTGRTTYYADSVKGRFTISRDNAKNTVYLQMNSLKPEDTAVYYCAAAPYGCSHYRDEYAYWGQGTQVTVSSHHHHHH

SEQ ID NO: 4 - protein, 129 aa, Artificial Sequence
  4NBX.B-biotin103 v149
QVQLQESGGGLAQAGGSLRLSCAASGRTFSRSPMAWFRQPPGKEREFVAAGSSTGRTTYYADSVKGRFTISRDNAKNTVYLQMNSLKPEDTAVYYCAAAPYGCARYRDEYAYWGQGTQVTVSSHHHHHH

SEQ ID NO: 5 - protein, 129 aa, Artificial Sequence
  4NBX.B-biotin103 v186
QVQLQESGGGLAQAGGSLRLSCAASGRTFSRSPMAWFRQPPGKEREFVAAGSSTGTTTYYADSVKGRFTISRDNAKNTVYLQMNSLKPEDTAVYYCAAAPLGCARKFAESAYWGQGTQVTVSSHHHHHH

SEQ ID NO: 6 - protein, 129 aa, Artificial Sequence
  4NBX.B-biotin103 v186_Fr
QVQLQESGGGLVQAGGSLRLSCAASGRTFSRSPMAWYRQPPGKEREFVAAGSSTGTTTYYADSVKGRFTISRDNAKNTVYLQMNSLKPEDTAVYYCAAAPLGCARKFAESAYWGQGTQVTVSSHHHHHH

SEQ ID NO: 7 - protein, 129 aa, Artificial Sequence
  4NBX.B-biotin103 v186_Fr +1
QVQLQESGGGLVQAGGSLRLSCAASGRTFSRSPMAWYRQPPGKEREFVAAGSSTGTTTYYADSVKGRFTISRDNAKNTVYLQMNSLKPEDTAVYYCAAAPLGACRKFAESAYWGQGTQVTVSSHHHHHH

SEQ ID NO: 8 - protein, 129 aa, Artificial Sequence
  4NBX.B-biotin103 v186_Fr -1
QVQLQESGGGLVQAGGSLRLSCAASGRTFSRSPMAWYRQPPGKEREFVAAGSSTGTTTYYADSVKGRFTISRDNAKNTVYLQMNSLKPEDTAVYYCAAAPLCAARKFAESAYWGQGTQVTVSSHHHHHH

SEQ ID NO: 9 - protein, 134 aa, Artificial Sequence
  2X89.A-WT
QVQLQESGGGSVQAGGSLRLSCAASGYTDSRYCMAWFRQAPGKEREWVARINSGRDITYYADSVKGRFTFSQDNAKNTVYLQMDSLEPEDTATYYCATDIPLRCRDIVAKGGDGFRYWGQGTQVTVSSHHHHHH

SEQ ID NO: 10 - protein, 134 aa, Artificial Sequence
  2X89.A_CCAA_I57C
QVQLQESGGGSVQAGGSLRLSCAASGYTDSRYAMAWFRQAPGKEREWVARINSGRDCTYYADSVKGRFTFSQDNAKNTVYLQMDSLEPEDTATYYCATDIPLRARDIVAKGGDGFRYWGQGTQVTVSSHHHHHH

SEQ ID NO: 11 - protein, 134 aa, Artificial Sequence
  2X89.A-CCAA-biotin57 v37
QVQLQESGGGLVQAGGSLRLSCAASGYTDSRYAMGWYRQAPGKEREWVARISAGRDCTYYADSVKGRFTFSQDNAKNTVYLQMDSLKPEDTAVYYCATDIPLQATLITAKGGDGFRYWGQGTQVTVSSHHHHHH

SEQ ID NO: 12 - protein, 134 aa, Artificial Sequence
  2X89.A-CCAA-biotin57 v42
QVQLQESGGGLVQAGGSLRLSCAASGYTDSQYAMGWYRQAPGKEREWVATISAGRDCTYYADSVKGRFTFSQDNAKNTVYLQMDSLKPEDTAVYYCATDIPLQATLITAKGGKGFQYWGQGTQVTVSSHHHHHH

SEQ ID NO: 13 - protein, 134 aa, Artificial Sequence
  2X89.A-CCAA-biotin57 v20
QVQLQESGGGLVQAGGSLRLSCAASGYTSSQYAMGWYRQAPGKEREWVATISAGRDCTYYADSVKGRFTFSQDNAKNTVYLQMDSLKPEDTAVYYCATDSPLQATLITAKGGKGFQYWGQGTQVTVSSHHHHHH

SEQ ID NO: 14 - protein, 134 aa, Artificial Sequence
  2X89.A-CCAA-biotin57 v5
QVQLQESGGGLVQAGGSLRLSCAASGYTSSQYAMGWYRQAPGKEREWVATISAGADCTYYADSVKGRFTFSQDNAKNTVYLQMDSLKPEDTAVYYCATDSPLQATLITAKGGKGFQYWGQGTQVTVSSHHHHHH

SEQ ID NO: 15 - protein, 118 aa, Artificial Sequence
  SA(core 16-133)-WT
GITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKV

SEQ ID NO: 16 - protein, 118 aa, Artificial Sequence
  SA(core 16-133) S45A/T90A/D128A
GITGTWYNQLGSTFIVTAGADGALTGTYEAAVGNAESRYVLTGRYDSAPATDGSGTALGWTVAWKNNYRNAHSAATWSGQYVGGAEARINTQWLLTSGTTEANAWKSTLVGHATFTKV

SEQ ID NO: 17 - protein, 141 aa, Artificial Sequence
  mSA_WT
HHHHHHSQDLASAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSDYKDDDDK

SEQ ID NO: 18 - protein, 141 aa, Artificial Sequence
  mSAS27A
HHHHHHSQDLASAEAGITGTWYNQSGATFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGSDYKDDDDK